SEQUENCE LISTING (14 sequences)

SEQ ID NO: 1 - protein, 121 aa, Griffithsia sp.
  MOD_RES (31)..(31): Any amino acid
SLTHRKFGGSGGSPFSGLSSIAVRSGSYLDXIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 2 - protein, 121 aa, Artificial Sequence
  Description of Artificial Sequence Synthetic polypeptide
SLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 3 - protein, 193 aa, Artificial Sequence
  Description of Artificial Sequence Synthetic polypeptide
MGGSSHHHHHHSSGLVPRGSLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQYSSSGGGGSGGGSSSGSHTTWMEWDREINNYTSLIHSLIEESQNQQEKNEQELL

SEQ ID NO: 4 - protein, 226 aa, Artificial Sequence
  Description of Artificial Sequence Synthetic polypeptide
MGSSHHHHHHSSGLVPRGSHMIEGRFSPLSSQSSACCFAYIARPLPRAHIKEYFYTSGKCSNPAVVFVTRKNRQVCANPEKKWVREYINSLEMSGGGGSGGGGSGSLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQY

SEQ ID NO: 5 - protein, 183 aa, Artificial Sequence
  Description of Artificial Sequence Synthetic polypeptide
MGGSSHHHHHHSSGLVPRGSLTHRKFGGSGGSPFSGLSSIAVRSGSYLDAIIIDGVHHGGSGGNLSPTFTFGSGEYISNMTIRSGDYIDNISFETNMGRRFGPYGGSGGSANTLSNVKVIQINGSAGDYLDSLDIYYEQYSSSGGGGSGGGGSSSSCNLHFCQLRCKSLGLLGKCAGSFCACV

SEQ ID NO: 6 - protein, 27 aa, Artificial Sequence
  Description of Artificial Sequence Synthetic peptide
CNLHFCQLRCKSLGLLGKCAGSFCACV

SEQ ID NO: 7 - protein, 16 aa, Artificial Sequence
  Description of Artificial Sequence Synthetic peptide
SSSGGGGSGGGSSSGS

SEQ ID NO: 8 - protein, 91 aa, Artificial Sequence
  Description of Artificial Sequence Synthetic polypeptide
MHHHHHHIEGRHTTWMEWDREINNYTSLIHSLIEESQNQQEKNEQELLSSSGGGGSGGGGSSSSCNLHFCQLRCKSLGLLGKCAGSFCACV

SEQ ID NO: 9 - protein, 37 aa, Artificial Sequence
  Description of Artificial Sequence Synthetic polypeptide
HTTWMEWDREINNYTSLIHSLIEESQNQQEKNEQELL

SEQ ID NO: 10 - protein, 27 aa, Artificial Sequence
  Description of Artificial Sequence Synthetic peptide
CNLHFCQLRCKSLGLLGKCAGSFCACV

SEQ ID NO: 11 - protein, 25 aa, Artificial Sequence
  Description of Artificial Sequence Synthetic peptide
MGSSHHHHHHSSGLVPRGSHMIEGR

SEQ ID NO: 12 - protein, 16 aa, Artificial Sequence
  Description of Artificial Sequence Synthetic peptide
SSSGGGGSGGGSSSGS

SEQ ID NO: 13 - protein, 10 aa, Artificial Sequence
  Description of Artificial Sequence Synthetic peptide
GGGGSGGGGS

SEQ ID NO: 14 - protein, 4 aa, Artificial Sequence
  Description of Artificial Sequence Synthetic peptide
WMEW

(19) United States
(12) Patent Application Publication
(10) Pub. No.: US 2011/0263485 A1
(43) Pub. Date: Oct. 27, 2011

(54) Bifunctional Griffithsin Analogs

(75) Inventors: Patricia J. LiWang, Merced, CA (US); Ioannis Kagiampakis, College Station, TX (US)

(73) Assignee: The Regents of the University of California

(21) Appl. No.: 12/957,261

(22) Filed: Nov. 30, 2010

Related U.S. Application Data
(60) Provisional application No. 61/265,497, filed on Dec. 1, 2009.
(60) Provisional application No. 61/288,796, filed on Dec. 21, 2009.

(51) Int. Cl. A61K 38/16; C12N 5/071; C07H 21/00; A61P 31/18; C07K 14/405; C07K 16/00

(52) U.S. Cl. 514/3.8; 530/350; 530/387.3; 514/1.1; 536/23.1; 536/23.53; 435/375

(57) ABSTRACT

The present disclosure provides chimeric proteins, protein combinations and other compositions comprising a gp120-binding protein such as Griffithsin. Also provided are methods of using the proteins, protein combinations or compositions to prevent or treat HIV infection.

### CROSS REFERENCE TO RELATED APPLICATIONS

This application claims the benefit under 35 U.S.C. §119(e) of U.S. Provisional Ser. Nos. 61/265,497, filed Dec. 1, 2009 and 61/288,796, filed Dec. 21, 2009, the contents of each of which are incorporated by reference in their entirety into the current disclosure.

### STATEMENT OF GOVERNMENT SUPPORT

This invention was made with government support under Grant No. R21 A1079777 awarded by the National Institutes of Health. The government has certain rights in this invention.

### FIELD OF INVENTION

The present disclosure generally relates to compositions and methods for preventing or treating HIV infections.

### BACKGROUND OF THE DISCLOSURE

Throughout and within this application various technical and patent literature are referenced either explicitly or by reference to an Arabic numeral. The bibliographic citations for the Arabic numeral citations are found after the experimental examples. The contents of these technical and patent citations are incorporated by reference into this application to more fully describe the state of the art to which this disclosure pertains.

In addition to the more established anti-HIV (human immunodeficiency virus) strategies such as inhibition of protease or reverse transcriptase, viral entry inhibition has great potential in the fight against HIV infection and related acquired immune deficiency syndrome (AIDS). Entry inhibition generally refers to the process of blocking infection by the HIV virus before it can fully infect a cell. Entry inhibition entails stopping HIV before it breaches the cell, either as a strategy to prevent infection altogether or to curtail infection of new cells in an HIV-positive individual. It can be a useful therapy to either prevent infection (as part of a so-called microbicide formulation) or to prevent new cells from getting infected in an HIV-positive individual. HIV entry inhibitors (also called fusion inhibitors) usually block one or more of the interactions between HIV envelope proteins (gp120 and gp41) and cell surface proteins (CD4, CCR5, CXCR4).

About 2.7 million people are infected with HIV each year, and women comprise 50% of the 33 million people living with AIDS [1]. In the developing world, effective prevention strategies are lacking, often because women have limited freedom in choosing sexual situations or in insisting on condom use. Therefore, the development of an anti-HIV microbicide is important. Properties that are desirable in a microbicide include the ability to effectively inhibit HIV infection at low concentrations, the ability to be applied topically on a regular basis without causing inflammation, stability to fluctuating temperatures, and inexpensive production.

Several strategies have been proven effective at HIV entry inhibition either in vitro or in vivo: binding to viral surface proteins gp120 and gp41, binding to human cell surface receptor CD4, and binding to human cell surface co-receptors CCR5 and CXCR4.

A microbicide is a composition that can be used to reduce the infectivity of microbes such as HIV. It can be formulated into a cream or gel and used to prevent sexual spread of HIV. For example, for use in developing countries, the microbicide needs to be inexpensive to produce, stable under high temperature, and active at the lower pH's in the urogenital tract. Some proteins do not have these properties, which is a disadvantage, even though the proteins may be effective in a lab environment. It is shown that the single peptide HIV inhibitor, T-20/Fuzeon®/enfuvirtide, requires high doses to be effective in human patients even though it has an IC₅₀ (50% inhibition) in the quite acceptable 2-20 nM range in in vitro assays (Root & Steger (2004) Current Pharm. Design 10:1805-25).

A major drawback of the existing microbicides is that although an inhibitor may work at a low concentration in vitro, much higher doses are needed to protect a macaque from infection in an in vivo assay (Lederman et al. (2004) Science 306:485-487; Veazey et al. (2005) Nature 438:99-102). Thus, a need exists to provide a safe and effective microbiocide to combat HIV infection and infectivity. This disclosure satisfies this need and provides related advantages as well.

### SUMMARY OF THE DISCLOSURE

It is discovered herein that the combination of a gp120 Griffithsin and a peptide selected from a gp41-binding protein, a CCR5-binding protein, a gp120-binding protein or another Griffithsin, either in the form of a chimeric polypeptide or as a mixed composition, is a potent inhibitor to HIV infection. Also discovered is that the combination of a gp120-binding protein and a gp41-binding protein, either in the form of a chimeric polypeptide or as a mixed composition, is a potent HIV infection inhibitor.

Thus, in one aspect, the disclosure provides an isolated chimeric polypeptide comprising, or alternatively consisting essentially of, or alternatively consisting of, a first portion comprising at least one of amino acid residues 11 to 101 of SEQ ID NO. 2, SEQ ID NO. 2 or a substantial homologue or biological equivalent of either one thereof and a second portion selected from a gp41-binding protein, a CCR5-binding protein, a gp120-binding protein, SEQ ID NO. 2, amino acid residues 11 to 101 of SEQ ID NO. 2 or a substantial homologue or biological equivalent of any one thereof.

Another aspect of the disclosure provides a composition comprising, or alternatively consisting essentially of, or alternatively consisting of, a first polypeptide comprising at least one of amino acid residues 11 to 101 of SEQ ID NO. 2, SEQ ID NO. 2, or a substantial homologue or biological equivalent of either one thereof and a second polypeptide selected from a gp41-binding protein, a CCR5-binding protein, a gp120-binding protein, SEQ ID NO. 2, amino acid residues 11 to 101 of SEQ ID NO. 2 a substantial homologue or biological equivalent of any one thereof.

In one aspect, the gp41-binding protein is selected from C37, C37Q652L, C34, C52L, T-2635, T20 or a substantial homologue or biological equivalent of any one thereof. In another aspect, the gp41-binding protein is T20 or a substantial homologue or biological equivalent thereof.

In one aspect, the CCR5-binding protein is selected from RANTES, P2-RANTES, PSC-RANTES, MIP-1α, MIP-1β, U83A, a CCR5 antibody or a substantial homologue or biological equivalent of any one thereof. In another aspect, the CCR5-binding protein is P2-RANTES or a biological equivalent to P2-RANTES. Biological equivalents of P2-RANTES include, for example, 5P12-RANTES, 5P14-RANTES or 6P4-RANTES.

In one aspect, the gp120-binding protein is cyanovirin-N(CVN), 12p1, CD4M33, CD4M47 or a substantial homologue or biological equivalent of any one thereof.

The disclosure also provides an isolated polypeptide comprising, or alternatively consisting essentially of, or alternatively consisting of, an amino acid sequence of SEQ ID NO. 3, 4 or 5, or an amino acid sequence having at least 80% identity to SEQ ID NO. 3, 4 or 5. Also provided is an isolated chimeric polypeptide comprising, or alternatively consisting essentially of, or alternatively consisting of, a first portion that is at least about 80% identical to SEQ ID NO. 2 and a second portion selected from a gp41-binding protein, a CCR5-binding protein or a gp120-binding protein or a substantial homologue or biological equivalent of any one thereof.

Also provided is a polynucleotide encoding for any of the above chimeric polypeptides, a DNA construct comprising an expression vector and the polynucleotide, or an isolated host cell transformed with the polynucleotide.

The disclosure further provides a method for preventing or inhibiting HIV entry into a cell or HIV replication in a cell capable of hosting HIV infection, comprising, or alternatively consisting essentially of, or yet further consisting of, contacting the cell with an effective amount of any of the above chimeric polypeptides or compositions. In one aspect, the chimeric polypeptides or compositions inhibit entry into the cell and in addition or alternatively, they act as fusion inhibitors. In some embodiments, the cell is an animal cell, such as a mammalian cell, e.g., a human cell. In one aspect of the disclosure, the cell is a human cell.

Further provided is a method for treating a subject in need thereof, comprising, or alternatively consisting essentially of, or alternatively consisting of, administering to the subject an effective amount of any of the above chimeric polypeptides or compositions. The subject can be a subject infected with HIV or a subject at risk of HIV infection. In some embodiments, the subject is an animal, a mammal, or a human. In some embodiments, administration of the chimeric polypeptides or composition is by injection or topical application.

The present disclosure further provides, in one embodiment, an isolated chimeric polypeptide comprising, or alternatively consisting essentially of or yet further consisting of a first portion comprising a gp41-binding protein and a second portion comprising a gp120-binding protein. In one aspect, the gp41-binding protein comprises, or alternatively consists essentially of, or yet further consists of, an anti-gp 41 antibody, fragment and derivative thereof, a C-peptide or a N-peptide examples of which include, but are not limited to an amino acid sequence comprising or alternatively consisting essentially of, or yet further consisting of one or more of C37, C37(Q652L), C37-ac, N-acetylated, C-term amidated C37 and N-acetylated, C-term amidated C37(Q652L), C34, C52L, T-2635, T20, N17, N23, N36, or a substantial homologue thereof. In a further aspect, gp41-binding protein comprises, or alternatively consists essentially of, or yet further consists of, an amino acid of one or more of C37, C-37ac, N-acetylated, C-term amidated C37 and N-acetylated, C-term amidated C37(Q652L) or a substantial homologue thereof. The substantial homologue is an amino acid sequence having greater than about 80% homology, or alternatively greater than about 80% homology, or alternatively greater than about 90% homology or alternatively greater than about 95% homology, or alternatively greater than about 98% homology, to the amino acid sequence of the respective peptide.

In one aspect, the gp120-binding protein comprises, or alternatively consists essentially of, or yet further consists of, an amino acid sequence comprising, or alternatively consisting essentially of, or yet further consisting of an anti-gp120 antibody, fragment or derivative thereof, actinohivin, cyanovirin-N(CVN), 12p1, CD4M33, CD4M47, CD4M33_{C1F23}, C37CD4M33_{C1F23} or a substantial homologue of any one thereof. In a particular aspect, the peptide known as Griffithsin is specifically excluded as a gp120-binding protein. The substantial homologue is an amino acid sequence having greater than about 80% homology, or alternatively greater than about 80% homology, or alternatively greater than about 90% homology or alternatively greater than about 95% homology, or alternatively greater than about 98% homology, to the amino acid sequence of the respective peptide.

In a further aspect, the gp120-binding protein further comprises, or alternatively consists essentially of, or yet further consists of a small molecule that binds to gp120, and example of which is metallocene.

Another aspect of the disclosure provides a peptide conjugate comprising, or alternatively consisting essentially of, or alternatively consisting of, a carrier covalently or non-covalently linked to an isolated chimeric polypeptide of the disclosure. In some embodiments, the carrier comprises a liposome, or alternatively a micelle, or alternatively a pharmaceutically acceptable polymer, or a carrier, e.g. a pharmaceutically acceptable carrier.

Yet another aspect of the disclosure provides an isolated polynucleotide encoding for an isolated chimeric polypeptide, an antibody, or a biologically active fragment of the antibody of the disclosure. Also provided is a DNA construct comprising an expression vector and a polynucleotide. In one aspect of the DNA construct, the vector is a plasmid vector, a yeast artificial chromosome, or a viral vector. In one aspect, the vector of the DNA construct comprises a protein tag. Protein tags can be selected from a GST-tag, a myc-tag, or a FLAG-tag provided in expression constructs commercially available from, e.g., Invitrogen, Carlsbad, Calif. Compositions comprising, or alternatively consisting essentially of, or yet further consisting of the isolated polynucleotides as described above and host cells as described are further provide by this disclosure.

The disclosure, in another aspect, provides an antibody that binds an isolated chimeric polypeptide of the disclosure. The antibody can be a polyclonal antibody, a monoclonal antibody, a chimeric antibody, a humanized antibody or a derivative or fragment thereof as defined below. In one aspect, the fragment comprises, or alternatively consists essentially of, or yet further consists of the CDR of the antibody. In one aspect, the antibody is detectably labeled or further comprises a detectable label conjugated to it. Also provided is a hybridoma cell line that produces a monoclonal antibody of this disclosure. Compositions comprising or alternatively consisting essentially of or yet further, consisting of one or more of the above embodiments are further provided herein.

The disclosure, in one aspect, provides a method for preventing or inhibiting HIV entry into a cell, comprising contacting the cell with an effective amount of an isolated chimeric polypeptide or an effective amount of a polynucleotide encoding the chimeric polypeptide of the disclosure. The contacting can be in vitro or in vivo. The cell can be an animal cell, a mammalian cell, or a human cell. In a particular aspect, the cell is a human cell.

### BRIEF DESCRIPTION OF THE DRAWINGS

FIG. 1A-G graphically illustrate the overall events of HIV-1 fusion into CD4⁺ T cells. In panel (A) the viral spike consists of a gp120 trimer and a gp41 trimer. Both of these proteins are heavily glycosylated. The human cells targeted by HIV have CD4 and chemokine (CCR5 and CXCR4) receptors on their surface. Panel (B) shows gp120 first interacts with CD4 on the surface of the cell. Panels (C) and (D) show that gp120-CD4 interaction induces conformational changes to gp120 and exposes cryptic epitopes that allow gp120 to interact with the chemokine receptor (co-receptor) CCR5 (or CXCR4). HIV gp41 is likely partially exposed after gp120 binds to CD4. In Panel (D), the fusion peptide of gp41 binds the target cell membrane. Panel (E) shows the domains of gp41 and Panel (F) shows the six helix bundle formation. The gp41 N-terminal heptad repeat folds back to form a “trimer of hairpins” with the C-terminal heptad repeat. This formation may bring the two membranes closer together or stabilize the pore. Panel (G) shows that C37 binds to the N-terminal segment of gp41 and blocks the formation of the 6-helix bundle.

FIG. 2A-B illustrate in general, one means by which the compositions block infection by HIV. This illustration involves stopping one of the processes illustrated in FIG. 1. For instance, as shown here, Griffithsin binds to sugars on the surface of gp120 and gp41, presumably inhibiting the binding of the virus to the cellular proteins (A). The peptide C37 (similar to C34 and T-20 and C52L) binds to the N-terminus of HIV gp41, stopping the 6-helix bundle formation (B).

FIG. 3 shows the results of a comparison of Grft-linker-C37 with Grit and with C37 in a cell-cell fusion assay using an R5-tropic effector cell. Also shown in this figure is that P2-RANTES (also shown as “P2”)-linker-Griffithsin (as well as P2-RANTES+Griffithsin in combination) are more effective than Griffithsin alone. A lower bar indicates a lower IC₅₀, which indicates more potent HIV inhibition.

FIG. 4 shows a comparison of Grft-linker-C37 with Grit and with C37 in a cell-cell fusion assay using an X4-tropic effector cell. As with R5 cells, in X4 tropic assays, Grft-linker-C37 is a better HIV inhibitor in terms of IC₅₀. In this case, combinations that include P2-RANTES (also shown as “P2”) are not expected to perform well, since P2-RANTES binds to CCR5, while by definition an “X4”: assay utilizes the CXC4 receptor, not CCR5. A lower bar indicates a lower IC₅₀, which indicates more potent HIV inhibition.

FIG. 5 shows that Grft-linker-C37 performs better than either Griffithsin alone or C37 alone (or the two in unlinked combination). Also, P2-RANTES (also shown as “P2”) with Griffithsin (either linked or unlinked) appears to perform better than either protein alone, although the unlinked compound appears to be more statistically significant and in general performs better than the linked P2-RANTES-linker-Grft.

FIG. 6 summarizes some of the results shown in the above figures and also shows data for the compound Grft-linker-CD4M33. The peptide CD4M33 was designed to bind to gp120 in the CD4-binding site, disallowing actual CD4 binding. Overall, Griffithsin-linker-CD4M33 is quite effective in both R5 and X4 cell fusion assays.

FIG. 7 shows the results of cell fusion assays. Panel (A) is a sample R5 fusion assay using ADA effector cells and P5L CCR5-bearing target cells. Panel (B) shows a sample X4 fusion assay using HL2/3 effector cells and TZM-bl CXCR4-bearing target cells. Each experiment was done at least 3 times in triplicate, and the results are presented as the average plus/minus the standard deviation.

FIG. 8 shows ¹⁵N-¹H correlation spectra of the HIV inhibitors. C37-linker-CD4M33_{C1F23}. Circled peaks on this spectrum indicate resonances that are consistent with folded protein.

FIG. 9A-C present ¹⁵N-¹H correlation spectra of some of the HIV inhibitors used in this study. A. Wild type griffithsin. B. Griff37 (griffithsin linked to C37). C. C37-linker-CD4M33_{C1F23}. Circled peaks on this spectrum indicate resonances that are consistent with folded protein in this type of spectrum.

### DETAILED DESCRIPTION OF THE DISCLOSURE

### I. Definitions

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

A “subject” of diagnosis or treatment is a cell or an animal such as a mammal, or a human. Non-human animals subject to diagnosis or treatment are those subject to HIV or similar virus (e.g., Simian Immunodeficiency Virus (SIV)) that include, for example, simians, murine, such as, rats, mice, canine, such as dogs, leporids, such as rabbits, livestock, sport animals, and pets.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. Single letter and three letter abbreviations of the naturally occurring amino acids are known in the art and “X” is used herein to indicate an unnatural or unidentified amino acid.

A “chimeric polypeptide”, “chimeric protein” or “fusion protein” refers to a protein, peptide or polypeptide created through the joining of two or more amino acid sequences or alternatively created by expression of a joint nucleotide sequence comprising two or more nucleotide sequences which originally code for separate proteins, peptides, polypeptides. Translation of joined nucleotide sequence, also known as a fusion gene, results in a single polypeptide, the “chimeric polypeptide”, with functional properties derived from each of the original proteins.

A “linker” or “peptide linker” refers to a peptide sequence linked to a polypeptide sequence at both ends of the linker peptide sequence. In one aspect, the linker is from about 1 to about 50 amino acid residues long or alternatively 1 to about 45, about 1 to about 40, about 1 to about 35, about 1 to about 30, about 1 to about 25, about 1 to about 20, about 1 to about 15, about 1 to about 10, about 1 to about 9, about 1 to about 8, about 1 to about 7, about 1 to about 6, about 1 to about 5, about 2 to about 40, about 2 to about 30, about 2 to about 25, about 2 to about 20, about 2 to about 15, about 2 to about 10, about 2 to about 9, about 2 to about 8, about 2 to about 7, about 2 to about 6, about 2 to about 5, about 3 to about 40, about 3 to about 30, about 3 to about 20, about 3 to about 15, about 3 to about 10, about 3 to about 9, about 3 to about 8, about 3 to about 7, about 3 to about 5, about 4 to about 40, about 4 to about 30, about 4 to about 20, about 4 to about 10, about 4 to about 8, about 4 to about 6, about 5 to about 40, about 5 to about 30, about 5 to about 20, about or 5 to about 10 amino acid residues long. In a particular aspect, the linker is from about 1 to about 20 amino acid residues long. In another particular aspect, the linker is from about 3 to 10 amino acid residues long.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

As used herein, the term “biological equivalent thereof” when referring to a reference protein, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid.

A biological equivalent of P2-RANTES includes without limitation 5P12-RANTES and 5P14-RANTES as well as those described in Gaertner et al. (2008) PNAS 105:17706-17711. PSC-RANTES is another biological equivalent of RANTES designed by Lederman et al. (2004) Science 306:485-7.

A polynucleotide or polynucleotide region (or a polypeptide or polypeptide region) having a certain percentage or identity (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by ═HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

A “substantial homologue” of a polynucleotide or polypeptide refers to a polynucleotide or a polypeptide having a substantial homology or identity to the polynucleotide or polypeptide. In one aspect, a “substantial homology” is greater than about 80% homology, or alternatively greater than about 80% homology, or alternatively greater than about 90% homology or alternatively greater than about 95% homology, or alternatively greater than about 98% homology.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

“An effective amount” refers to the amount of an active chimeric polypeptide or a pharmaceutical composition sufficient to induce a desired biological and/or therapeutic result. That result can be alleviation of the signs, symptoms, or causes of a disease, or any other desired alteration of a biological system. In the present disclosure, the result will typically involve prevention or inhibition of HIV infection (or SIV if appropriate) or alleviation of signs or symptoms of HIV or SIV infection. The effective amount will vary depending upon the health condition or disease stage of the subject being treated, timing of administration of the chimeric polypeptide, the manner of administration and the like, all of which can be determined readily by one of ordinary skill in the art.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

“Treating” also covers any treatment of a disorder in a mammal, and includes: (a) preventing a disorder from occurring in a subject that may be predisposed to a disorder, but may have not yet been diagnosed as having it, e.g., prevent HIV or SIV infection to a subject at risk of HIV or SIV infection or prevent HIV or SIV infection to a healthy cell in a subject; (b) inhibiting a disorder, i.e., arresting its development, e.g., inhibiting HIV infection; or (c) relieving or ameliorating the disorder, e.g., reducing HIV or SIV infection.

As used herein, to “treat” further includes systemic amelioration of the symptoms associated with the pathology and/or a delay in onset of symptoms. Clinical and sub-clinical evidence of “treatment” will vary with the pathology, the subject and the treatment.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

The agents and compositions of the present disclosure can be used in the manufacture of medicaments and for the treatment of humans and other animals by administration in accordance with conventional procedures, such as an active ingredient in pharmaceutical compositions.

An agent of the present disclosure can be administered for therapy by any suitable route of administration. It will also be appreciated that the preferred route will vary with the condition and age of the recipient, and the disease being treated.

The term “conjugated moiety” refers to a moiety that can be added to an isolated chimeric polypeptide by forming a covalent bond with a residue of chimeric polypeptide. The moiety may bond directly to a residue of the chimeric polypeptide or may form a covalent bond with a linker which in turn forms a covalent bond with a residue of the chimeric polypeptide.

A “peptide conjugate” refers to the association by covalent or non-covalent bonding of one or more polypeptides and another chemical or biological compound. In a non-limiting example, the “conjugation” of a polypeptide with a chemical compound results in improved stability or efficacy of the polypeptide for its intended purpose. In one embodiment, a peptide is conjugated to a carrier, wherein the carrier is a liposome, a micelle, or a pharmaceutically acceptable polymer.

“Liposomes” are microscopic vesicles consisting of concentric lipid bilayers. Structurally, liposomes range in size and shape from long tubes to spheres, with dimensions from a few hundred Angstroms to fractions of a millimeter. Vesicle-forming lipids are selected to achieve a specified degree of fluidity or rigidity of the final complex providing the lipid composition of the outer layer. These are neutral (cholesterol) or bipolar and include phospholipids, such as phosphatidylcholine (PC), phosphatidylethanolamine (PE), phosphatidylinositol (PI), and sphingomyelin (SM) and other types of bipolar lipids including but not limited to dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components are phospholipids, such as hydrogenated soy phosphatidylcholine (HSPC), lecithin, phosphatidylethanolamine, lysolecithin, lysophosphatidylethanol-amine, phosphatidylserine, phosphatidylinositol, sphingomyelin, cephalin, cardiolipin, phosphatidic acid, cerebrosides, distearoylphosphatidylethan-olamine (DSPE), dioleoylphosphatidylcholine (DOPC), dipalmitoylphosphatidylcholine (DPPC), palmitoyloleoylphosphatidylcholine (POPC), palmitoyloleoylphosphatidylethanolamine (POPE) and dioleoylphosphatidylethanolamine 4-(N-maleimido-methyl)cyclohexane-1-carb-oxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, and the cationic lipids mentioned above (DDAB, DODAC, DMRIE, DMTAP, DOGS, DOTAP (DOTMA), DOSPA, DPTAP, DSTAP, DC-Chol). Negatively charged lipids include phosphatidic acid (PA), dipalmitoylphosphatidylglycerol (DPPG), dioleoylphosphatidylglycerol and (DOPG), dicetylphosphate that are able to form vesicles. Typically, liposomes can be divided into three categories based on their overall size and the nature of the lamellar structure. The three classifications, as developed by the New York Academy Sciences Meeting, “Liposomes and Their Use in Biology and Medicine,” December 1977, are multi-lamellar vesicles (MLVs), small uni-lamellar vesicles (SUVs) and large uni-lamellar vesicles (LUVs).

A “micelle” is an aggregate of surfactant molecules dispersed in a liquid colloid. A typical micelle in aqueous solution forms an aggregate with the hydrophilic “head” regions in contact with surrounding solvent, sequestering the hydrophobic tail regions in the micelle center. This type of micelle is known as a normal phase micelle (oil-in-water micelle). Inverse micelles have the head groups at the center with the tails extending out (water-in-oil micelle). Micelles can be used to attach a polynucleotide, polypeptide, antibody or composition described herein to facilitate efficient delivery to the target cell or tissue.

The phrase “pharmaceutically acceptable polymer” refers to the group of compounds which can be conjugated to one or more polypeptides described here. It is contemplated that the conjugation of a polymer to the polypeptide is capable of extending the half-life of the polypeptide in vivo and in vitro. Non-limiting examples include polyethylene glycols, polyvinylpyrrolidones, polyvinylalcohols, cellulose derivatives, polyacrylates, polymethacrylates, sugars, polyols and mixtures thereof.

“Pharmaceutically acceptable carriers” refers to any diluents, excipients, or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide of this disclosure can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmic vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacteria produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearised by using restriction enzymes, and then DNA ligase can add a sequence or gene of interest within the linear molecule by the use of cohesive ends. Yeast expression vectors, such as YACs, Ylps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat & Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this disclosure. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this disclosure are other non-limiting techniques.

An example of a solid phase support include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. A eukaryotic host, including, for example, yeast, higher plant, insect and mammalian cells. Non-limiting examples include simian, bovine, porcine, murine, rats, avian, reptilian and human.

“Prokaryotic cells” that usually lack a nucleus or any other membrane-bound organelles and are divided into two domains, bacteria and archaea. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to bacillus bacteria, E. coli bacterium, and Salmonella bacterium.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The antibodies can be polyclonal or monoclonal and can be isolated from any suitable biological source, e.g., murine, rat, sheep and canine.

The term “human antibody” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

As used herein, “isotype” refers to the antibody class (e.g., IgM or IgG1) that is encoded by heavy chain constant region genes.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

The terms “monoclonal antibody” or “monoclonal antibody composition” as used herein refer to a preparation of antibody molecules of single molecular composition. A monoclonal antibody composition displays a single binding specificity and affinity for a particular epitope.

As used herein, the term “label” intends a directly or indirectly detectable compound or composition that is conjugated directly or indirectly to the composition to be detected, e.g., N-terminal histadine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BIue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

“Griffithsin”, or “GRFT”, is a protein isolated from the red algae Griffithsia. It has a 121-amino acid sequence and has been shown in vitro to be a highly potent HIV entry inhibitor (Mori et al. (2005) J. Biol. Chem. 280(10):9345-53). Griffithsin is currently being investigated as a potential microbicide for use in the prevention of the transmission of HIV (Emau et al. (2007) J. Med. Primatol. 36(4-5):244-53). It has recently been reported that Griffithsin can be produced inexpensively in large quantities from tobacco leaves (O'Keefe et al. (2009) Proc. Nat. Acad. Sci. USA 106(15):6099-6104) and in E. coli (Giomarelli et al. (2006) Protein Exp. and Purification 47:194-202).

The natural Griffithsin protein sequence includes an unnatural amino acid at position 31 (see Swiss-Prot entry P84801 and SEQ ID NO. 1 in Table 1). Naturally occurring Griffithsin is a 121 amino acid protein with a mostly beta-sheet structure. This protein has been shown in crystal structures to form a “domain-swapped dimer” in which two beta strands from one domain are incorporated into a beta sheet composed mostly of strands from the other domain. Griffithsin appears to have three saccharide binding sites per monomer, for a total of 6 sites in the dimer. The three sites are in the following areas: site 1 is in the region around Asp112, site 2 is around the area of Asp30 and site 3 is around the area of Asp70. Each site is also comprised of many amino acids that may provide H-bonds, structural support or indirect contacts to the saccharide. Ziolkowska et al. (2006) Structure 14:1127-1135. Substitution of the unnatural amino acid with an alanine does not impact the function of Griffithsin (see SEQ ID NO. 2 in Table 2). It is also known that removal or substitution of about 10 amino acid residues from each terminus of the Griffithsin protein does not substantially impact the activity of the protein. Applicants have further determined that the addition of an N-terminal “G” and/or N-terminal histidine tag (N-his tag) to the Griffithsin fragments do not impact the function and these amino acids are included within the scope of this disclosure.

“Fusion inhibitors” or “entry inhibitors” as used herein interchangeably and refer to a class of antiretroviral drugs, used in combination therapy for the treatment of HIV infection and the like. This class of drugs interferes with one or more of the binding, fusion and entry of an HIV virion to a human cell. By blocking these steps in HIV's replication cycle, such agents inhibit or slow HIV infection. There are several key proteins involved in the HIV entry process: CD4, a protein receptor found on the surface of helper T cells in the human immune system, also called CD4+ T cells; gp120, a protein on HIV surface that binds to the CD4 receptor; CCR5, a second receptor found on the surface of CD4+ cells, called a chemokine co-receptor; CXCR4, another chemokine co-receptor found on CD4+ cells; and gp41, a HIV protein, closely associated with gp120, that penetrates the cell membrane.

A “gp41-binding protein” or “gp41-binding peptide” refers to a protein that binds to the gp41 protein. HIV binds to the host CD4+ cell receptor via the viral protein GP120; GP41, a viral transmembrane protein, then undergoes a conformational change that assists in the fusion of the viral membrane to the host cell membrane. Non-limiting examples of gp41-binding proteins include C-peptides, C37, C34, C52L, T-2635 and T20.

“C-peptides”, or “synthetic C peptides” refer to peptides that are derived from the C-terminal heptad repaeat of the HIV type 1 (HIV-1) gp41 envelope protein. These C-peptides bind to the N-terminal heptad repeat of gp41 and inhibit HIV fusion. One C-peptide (T-20, also called Fuzeon or enfuvirtide from Trimeris/Roche) is currently in clinical use. The drawbacks of these peptides are that they generally require relatively high doses when in clinical use. And even in in vitro studies, they have been surpassed in terms of low concentrations needed for effectiveness by both griffithsin alone and variants of CC chemokines. But they are still considered to be a very clinically useful class of drugs.

“CC chemokines” or “β-chemokines” are chemokines that have two adjacent cysteines near their amino terminus. There have been at least 27 distinct members of this subgroup reported for mammals, called CC chemokine ligands (CCL)-1 to -28; CCL10 is the same as CCL9. Chemokines of this subfamily usually contain four cysteines (C4-CC chemokines), but a small number of CC chemokines possess six cysteines (C6-CC chemokines). An example of a CC chemokine is monocyte chemoattractant protein-1 (MCP-1 or CCL2) which induces monocytes to leave the bloodstream and enter the surrounding tissue to become tissue macrophages. CC chemokines induce cellular migration by binding to and activating CC chemokine receptors, ten of which have been discovered to date and called CCR1-10. These receptors are expressed on the surface of different cell types allowing their specific attraction by the chemokines.

“C37” is a peptide that is derived from C34 which in turn is a sequence in gp41 that binds to the N-terminal region of gp41 to stop the 6 helix bundle formation. It has the sequence HTTWMEWDREINNYTSLIHSLIEESQNQQEKNEQELL that is derived from HIV-1. HIV-1-HXB2 residues 625 to 6161 contains the entire C34 sequence (W628 to L661) as reported by Root et al. (2001) Science 291:884-888.

“C37-ac” is C-terminally acetylated, N-terminally amidated C37 peptide and is intended to be included in the use of the term “C37”. That is, the two ends are “capped” so that they are not charged.

“T20” or “Enfuvirtude” is an HIV fusion inhibitor, marketed under the trade name Fuzeon®. Without being bound by theory, T20 is believed to work by disrupting the HIV-1 molecular machinery at the final stage of fusion with the target cell, preventing uninfected cells from becoming infected. A biomimetic peptide, enfuvirtide was rationally designed to mimic components of the HIV-1 fusion machinery and displace them, preventing normal fusion. Enfuvirtide binds to GP41 preventing the creation of an entry pore for the capsid of the virus, keeping it out of the cell. Generally, C-peptides can inhibit HIV-1 membrane fusion by binding to the amino-terminal trimeric coiled coil of the same protein. T-20 contains an additional tryptophan-rich sequence motif whose binding site extends beyond the gp41 coiled-coil region yet provides the key determinant of inhibitory activity in T-20.

“034” is a synthetic C-peptide composed of a peptide sequence that overlaps with T-20 but contains the gp41 coiled-coil cavity binding residues, ⁶²⁸WMEW⁶³¹. C34 is known to compete with the CHR of gp41 for the hydrophobic grooves of the NHR yet is incapable of functioning at a post-lipid mixing stage (Stoddart et al. (2008) J. Biol. Chem. 283(49):34045-52).

“C52L” is a recombinant peptide inhibitor that includes both the C-peptide and tryptophan-rich regions of T-20 (Deng (2007) Biochemistry 46(14):4360-9). The C52L peptide potently inhibits in vitro infection of human T cells. C52L can be expressed in bacteria so it might be more economical to manufacture on a large scale than T-20-like peptides produced by chemical synthesis.

“T-2635” is a helix-stabilized second generation fusion inhibitor with antiviral activity against virus strains resistant to enfuvirtide. It was designed by Dwyer et al. (Dwyer et al. (2007) Proc. Natl. Acad. Sci. USA 104(31):12772-7).

A “CCR5-binding protein” refers to a protein that binds to CCR5. CCR5, short for chemokine (C—C motif) receptor 5, is a protein which in humans is encoded by the CCR5 gene which is located on chromosome 3 on the short (p) arm at position 21. CCR5 has also recently been designated CD195 (cluster of differentiation 195). The CCR5 protein functions as a chemokine receptor in the CC chemokine group. The natural chemokine ligands that bind to this receptor are RANTES, MIP-1α and MIP-1β. CCR5 is predominantly expressed on T cells, macrophages, dendritic cells and microglia. It is likely that CCR5 plays a role in inflammatory responses to infection, though its exact role in normal immune function is unclear. Non-limiting examples of CCR5-binding proteins include RANTES, P2-RANTES, MIP-1α, MIP-1β, U83A and CCR5 antibodies.

“RANTES”, “Chemokine (C—C motif) ligand 5” or simply “CCL5” refers to a protein which in humans is encoded by the CCL5 gene. CCL5 is an 8 kDa protein classified as a chemotactic cytokine or chemokine. CCL5 is chemotactic for T cells, eosinophils, and basophils, and plays an active role in recruiting leukocytes into inflammatory sites. With the help of particular cytokines (i.e., IL-2 and IFN-γ) that are released by T cells, CCL5 also induces the proliferation and activation of certain natural-killer (NK) cells to form CHAK (CC-Chemokine-activated killer) cells. It is also an HIV-suppressive factor released from CD8+ T cells. This chemokine has been localized to chromosome 17 in humans. RANTES was first identified in a search for genes expressed “late” (3-5 days) after T cell activation. It was subsequently determined to be a CC chemokine and expressed in more than 100 human diseases. RANTES expression is regulated in T lymphocytes by Kruppel like factor 13 (KLF13). RANTES was earlier called Regulated upon Activation, Normal T-cell Expressed, and Secreted, abbreviated RANTES. There have been many variants of the CC chemokine RANTES (and some of other CC chemokines) that have been shown to have strong anti-HIV activity. Several of these have been patented including P2-RANTES (Hartley et al. (2003) J. Virol. 77:637-644) and PSC-RANTES. Most recently, next generation RANTES variants have been isolated by random mutagenesis, and these have the combination of high effectiveness (at low (pM) concentration) as well as being able to be produced from E. coli. The publication which introduced P2-RANTES is Hartley et al. (2003) J. Virol. 77:637-644. Some other RANTES variants are shown in Gaertner et al. (2008) PNAS105:17706-17711. “PSC-RANTES” is another RANTES variant designed by Lederman et al. (Lederman et al. (2004) Science 306:485-7).

“MIP-1α” or “macrophage inflammatory protein-1α”, and “MIP-1β” or “macrophage inflammatory protein-1β” are from the CC (cysteine-cysteine) chemokine subfamily. These soluble factors are chemotactic for specific types of leukocyte populations and are involved in the regulation of cell-mediated immunity. MIP-1α and MIP-1β are produced by monocytes, macrophages, lymphocytes, and other cell types (see generally, e.g., Matsukawa et al. (2000) Chemokines and innate immunity. Rev. Immunogenet. 2:339-358). MIP-1α and MIP-1β have been shown to inhibit HIV entry by binding to the co-receptor CCR5.

“U83A” is a distant chemokine homolog encoded by a human herpesvirus-6 variant (Dewin & Gompels (2006) J. Immunol. 176(1):544-56). U83A can efficiently and potently induce calcium mobilization in cells expressing single human CCR1, CCR4, CCR6, or CCR8. U83A can also induce chemotaxis of Th2-like leukemic cells expressing CCR4 and CCR8.

A “gp120-binding protein” refers to a protein that binds gp120. gp120 is a glycoprotein exposed on the surface of the HIV envelope that binds to the CD4 receptor. gp120 is essential for virus entry into cells as it plays a vital role in seeking out specific cell surface receptors for entry. The crystal structure of gp120 complexed to D1 D2 CD4 and a neutralizing antibody Fab was solved in 1998. It is organized with an outer domain, an inner domain with respect to its termini and a bridging sheet. The gp120 gene is around 1.5 kb long and codes for around 500 amino acids. Three gp120s, bound as heterodimers to a transmembrane glycoprotein, gp41, are thought to combine in a trimer to form the envelope spike, which is involved in virus-cell attachment. Non-limiting examples of gp120-binding proteins include Griffinthsin, cyanovirin-N(CVN), 12p1, CD4M33 and CD4M47.

The terms “cyanovirin-N” and “CVN” refer to an 11 kD protein originally isolated from the cyanobacteria Nostoc ellipsosporum (Boyd et al. (1997) Antimicrob. Agents Chemother. 41(7):1521-30). CNV inactivates a broad range of clades of HIV-1, SIV, and FIV, and prevents cell to cell transmission of infection. Recent investigations using both in vitro and in vivo assays yield support for the efficacy of CV-N as a microbicidal candidate.

The term “12p1” refers to the linear peptide 12p1 which was initially isolated from a phage display library and found to inhibit interaction of HIV-1 gp120 with both CD4 and a CCR5 surrogate, mAb 17b (Ferrer & Harrison (1997) J. Virol. 73(7):4795-5801). There is a direct interaction of 12p1 with gp120, which occurs with a binding stoichiometry of 1:1. The peptide was shown to inhibit the binding of monomeric YU2 gp120 to both sCD4 and 17b. Peptide 12p1 also inhibited binding of these ligands to trimeric envelope glycoproteins, blocked the binding of gp120 to the native co-receptor CCR5, and specifically inhibited HIV-1 infection of target cells in vitro. Analyses of sCD4 saturation of monomeric gp120 in the presence or absence of a fixed concentration of peptide suggest that 12p1 suppression of CD4 binding to gp120 is due to allosteric inhibitory effects rather than competitive inhibition of CD4 binding. Using a panel of gp120 mutants that exhibit weakened inhibition by 12p1, the putative binding site of the peptide was mapped to a region immediately adjacent to, but distinguishable from, the CD4 binding footprint. 12p1 was unable to inhibit binding of sCD4 to a gp120 mutant, S375W, which is believed to resemble the CD4-induced conformation of gp120. The results obtained to date strongly suggest that 12p1 preferentially binds gp120 prior to engagement of CD4, and alters the conformational state of gp120 to a form that has suppressed interactions with receptor ligands (CD4 and CCR5/CXCR4) that are generally believed crucial for viral entry.

“CD4M33” is a 27 amino acid peptide designed by Martin et al. to be a mimic of the human protein CD4 with optimized interactions to HIV gp120 (Martin et al. (2003) Nature Biotechnology 21:71-76). It was found to bind to gp120 and inhibit HIV entry. “CD4M47” is a derivative and biological equivalent of CD4M33 optimized at four positions (Stricher et al. (2008) J. Mol. Biol. 382(2):510-24).

### Descriptive Embodiments

A microbicide is a composition that can be used to reduce the infectivity of microbes such as HIV. It can be formulated into a cream or gel and used to prevent sexual spread of HIV. For example, for use in developing countries, the microbicide needs to be inexpensive to produce, stable under high temperature, and active at the lower pH's in the urogenital tract. This disclosure satisfies these needs and provides related advantages as well.

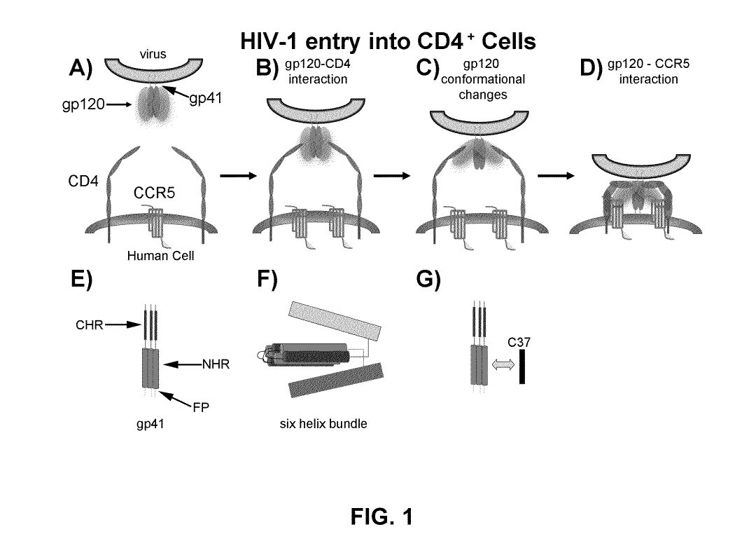

The early events in HIV infection are diagrammed in FIG. 1. The HIV envelope protein gp120 first makes contact with the human cell surface protein CD4 (FIG. 1B), which causes a conformational change in gp120 (FIG. 1C). The gp120-CD4 interaction facilitates the formation and exposure of the binding site on gp120 for its co-receptor on the human cell, the chemokine receptor CCR5 (or CXCR4 in some strains) (FIG. 1D) (Berger et al. (1999) Annu. Rev. Immunol. 17:657-700; Kwong et al. (1998) Nature 393:648-59; Sattentau et al. (1993) J. Virol. 67:7383-93; Sullivan et al. (1998) J. Virol. 72:4694-703). These HIV-cell interactions lead to the exposure of HIV protein gp41, which mediates cell fusion. Gp41 exists as a trimer having three major segments: The N-terminal fusion peptide (FP) which inserts into the cell, the so-called N-terminal heptad repeat, and the C-terminal heptad repeat (FIG. 1E). After the fusion peptide has inserted into the cell membrane, the N-terminal segment and C-terminal segment come together to form a 6 helix bundle, a trimer of hairpins (reviewed in (Eckert & Kim (2001) Annu. Rev. Biochem. 70:777-810; Root & Steger (2004) Curr. Pharm. Des. 1805-25) (FIG. 1F). This action has the effect of pulling the viral membrane surface close to the cellular surface, facilitating the formation or stabilization of a viral pore. It has recently been reported that these events may in part occur in the endosome: early binding events in cell fusion may occur the cell surface, after which the entire complex is internalized into an endosome for the final fusion process (Miyauchi et al. (2009) Cell 137:433-44).

It is discovered herein that the combination of a gp120 Griffithsin and a peptide selected from a gp41-binding protein, a CCR5-binding protein, a gp120-binding protein or another Griffithsin, either in the form of a chimeric polypeptide or as a mixed composition, is a potent inhibitor to HIV infection. Also discovered is that the combination of a gp120-binding protein and a gp41-binding protein, either in the form of a chimeric polypeptide or as a mixed composition, is a potent HIV infection inhibitor.

### Polypeptides and Compositions

One aspect of the present disclosure provides an isolated chimeric polypeptide comprising, or alternatively consisting essentially of, or alternatively consisting of, a first portion comprising amino acid residues 11 to 101 of SEQ ID NO. 2 SEQ ID NO. 2 or a substantial homologue or biological equivalent of either one thereof, and a second portion selected from a gp41-binding protein, a CCR5-binding protein, a gp120-binding protein amino acid residues 11 to 101 of SEQ ID NO. 2, SEQ ID NO. 2 or a substantial homologue or biological equivalent of any one thereof.

In some embodiments, the first portion is N-terminal to the second portion. Alternatively, the first portion is C-terminal to the second portion.

Also provided is an isolated chimeric polypeptide comprising, or alternatively consisting essentially of or yet further consisting of a first portion comprising a gp120 binding peptide and a second portion comprising a gp41 binding peptide. In one aspect, the gp41-binding peptide comprises, or alternatively consists essentially of, or yet further consists of, an amino acid sequence of one or more of a N-peptide, a C-peptide, an anti-gp41 antibody, fragment or derivative thereof, C37, C37-ac, C37(Q652L), C34, C52L, T-2635, T20, N17, N23, N36 or a substantial homologue thereof (see Eckert et al. (2001) PNAS 98(20):11187-11192). In a further aspect, gp41-binding peptide comprises, or alternatively consists essentially of, or yet further consists of, an amino acid of one or more of C37, C-37ac, C37 terminally amidated, C37(Q652L) or a substantial homologue thereof. The substantial homologue is an amino acid sequence having greater than about 80% homology, or alternatively greater than about 80% homology, or alternatively greater than about 90% homology or alternatively greater than about 95% homology, or alternatively greater than about 98% homology, to the amino acid sequence of the respective gp41-binding peptide.

In a further aspect, the isolated chimeric polypeptide as described above comprises an gp120-binding peptide that comprises, or alternatively consists essentially of, or yet further consist of, an anti-gp120 antibody, antibody fragment or derivative thereof, an amino acid sequence of actinohivin, cyanovirin-N(CVN), 12p1, CD4M33, CD4M47, CD4M33_{C1F23}, C37CD4M33_{C1F23} or a substantial homologue of any one thereof. In a further aspect, the gp120-binding peptide comprises, or alternatively consists essentially of, or yet further consist of, an amino acid sequence of CD4M33, CD4M47, CD4M33_{C1F23}, C37CD4M33_{C1F23} or a substantial homologue of any one thereof. In a particular aspect, the peptide known as Griffithsin is specifically excluded as a gp120-binding peptide.

In each of these embodiments, the substantial homologue is an amino acid sequence having greater than about 80% homology, or alternatively greater than about 80% homology, or alternatively greater than about 90% homology or alternatively greater than about 95% homology, or alternatively greater than about 98% homology to the respective gp41-binding peptide or gp120-binding peptide.

The first portion may be N-terminal to the second portion or alternatively, C-terminal to the second portion.

In some embodiments, the isolated chimeric polypeptide further comprises a peptide linker between the first portion and the second portion. In one aspect, the linker is from about 1 to about 50 amino acid residues long or alternatively about 1 to about 45, about 1 to about 40, about 1 to about 35, about 1 to about 30, about 1 to about 25, about 1 to about 20, about 1 to about 15, about 1 to about 10, about 1 to about 9, about 1 to about 8, about 1 to about 7, about 1 to about 6, about 1 to about 5, about 2 to about 40, about 2 to about 30, about 2 to about 25, about 2 to about 20, about 2 to about 15, about 2 to about 10, about 2 to about 9, about 2 to about 8, about 2 to about 7, about 2 to about 6, about 2 to about 5, about 3 to about 40, about 3 to about 30, about 3 to about 20, about 3 to about 15, about 3 to about 10, about 3 to about 9, about 3 to about 8, about 3 to about 7, about 3 to about 5, about 4 to about 40, about 4 to about 30, about 4 to about 20, about 4 to about 10, about 4 to about 8, about 4 to about 6, about 5 to about 40, about 5 to about 30, about 5 to about 20, about or 5 to about 10 amino acid residues long. In a particular aspect, the linker is from about 1 to about 20 amino acid residues long. In another particular aspect, the linker is from about 3 to about 10 amino acid residues long. In one aspect, the peptide linker is a polypeptide that comprises one or more amino acids, wherein at least one or more is selected from alanine, glycine or serine.

In some embodiments, the isolated chimeric polypeptide further comprises at least one of a protein start site, a polyhistidine tag, and/or a protease cleavage site, each operatively linked to the isolated chimeric polypeptide. Methods of designing, selecting and using protein start, polyhistidine tags, protease cleavage site in a protein expression system and operatively linking them to an expression target are known in the art. See, generally, Baneyx (2004) Protein Expression Technologies: Current Status and Future Trends, Taylor & Francis, 1^{st} Ed.

Another aspect of the disclosure provides an isolated polypeptide comprising, or alternatively consisting essentially of, or alternatively consisting of, an amino acid sequence of SEQ ID NO. 3, 4 or 5, or a substantial homologue or biological equivalent thereof. Further provided is an isolated chimeric polypeptide comprising, or alternatively consisting essentially of, or alternatively consisting of, a first portion that is at least about 80% identical to SEQ ID NO. 2 and a second portion selected from a gp41-binding protein, a CCR5-binding protein, a gp120-binding protein or a substantial homologue or biological equivalent of any one thereof.

For any chimeric polypeptide of the disclosure, the gp41-binding protein can be selected from C37, C34, C52L, T-2635, T20 or a substantial homologue or biological equivalent of any one thereof. In one aspect, the gp41-binding protein is T20 or a substantial homologue or biological equivalent thereof.

For any chimeric polypeptide of the disclosure, the CCR5-binding protein can be selected from RANTES, P2-RANTES, PSC-RANTES, MIP-1α, MIP-1β, U83A, a CCR5 antibody or a substantial homologue or biological equivalent of any one thereof. In one aspect, the CCR5-binding protein is P2-RANTES or a substantial homologue or biological equivalent thereof.

For any chimeric polypeptide of the disclosure, the gp120-binding protein can be cyanovirin-N(CVN), 12p1, CD4M33, CD4M47 or a substantial homologue or biological equivalent of any one thereof.

Polypeptides comprising the amino acid sequences of the disclosure can be prepared by expressing polynucleotides encoding the polypeptide sequences of this disclosure in an appropriate host cell. This can be accomplished by methods of recombinant DNA technology known to those skilled in the art. Accordingly, this disclosure also provides methods for recombinantly producing the polypeptides of this disclosure in a eukaryotic or prokaryotic host cells, as well as the isolated host cells used to produce the proteins. The proteins and polypeptides of this disclosure also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this disclosure also provides a process for chemically synthesizing the proteins of this disclosure by providing the sequence of the protein and reagents, such as amino acids and enzymes and linking together the amino acids in the proper orientation and linear sequence.

It is known to those skilled in the art that modifications can be made to any peptide to provide it with altered properties. Polypeptides of the disclosure can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that α-helical secondary structure or random secondary structure is preferred.

In a further embodiment, subunits of polypeptides that confer useful chemical and structural properties will be chosen. For example, peptides comprising D-amino acids may be resistant to L-amino acid-specific proteases in vivo. Modified compounds with D-amino acids may be synthesized with the amino acids aligned in reverse order to produce the peptides of the disclosure as retro-inverso peptides. In addition, the present disclosure envisions preparing peptides that have better defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂NH—R₂, where R₁, and R₂ are amino acid residues or sequences. A reduced peptide bond may be introduced as a dipeptide subunit. Such a molecule would be resistant to peptide bond hydrolysis, e.g., protease activity. Such molecules would provide ligands with unique function and activity, such as extended half-lives in vivo due to resistance to metabolic breakdown, or protease activity. Furthermore, it is well known that in certain systems constrained peptides show enhanced functional activity (Hruby (1982) Life Sciences 31:189-199 and Hruby et al. (1990) Biochem J. 268:249-262); the present disclosure provides a method to produce a constrained peptide that incorporates random sequences at all other positions.

Non-classical amino acids may be incorporated in the peptides of the disclosure in order to introduce particular conformational motifs, examples of which include without limitation: 1,2,3,4-tetrahydroisoquinoline-3-carboxylate (Kazrnierski et al. (1991) J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski & Hruby (1991) Tetrahedron Lett. 32(41):5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1989) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (1993) Int. J. Pep. Protein Res. 42(1):68-77) and (Dharanipragada et al. (1992) Acta. Crystallogr. C. 48:1239-1241).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Lett. 29:5057-5060); α-helix inducing analogs (Kemp et al. (1988) Tetrahedron Lett. 29:4935-4938); α-turn inducing analogs (Kemp et al. (1989) J. Org. Chem. 54:109:115); analogs provided by the following references: Nagai & Sato (1985) Tetrahedron Lett. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Clones et al. (1988) Tetrahedron Lett. 29:3853-3856); tetrazole (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 56:436. Conformationally restricted mimetics of beta turns and beta bulges, and peptides containing them, are described in U.S. Pat. No. 5,440,013, issued Aug. 8, 1995 to Kahn.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobicity, size, or charge. Methods used to determine the appropriate amino acid to be substituted and for which amino acid are know to one of skill in the art. Non-limiting examples include empirical substitution models as described by Dahoff et al. (1978) In Atlas of Protein Sequence and Structure Vol. 5 suppl. 2 (ed. M. O. Dayhoff), pp. 345-352. National Biomedical Research Foundation, Washington D.C.; PAM matrices including Dayhoff matrices (Dahoff et al. (1978), supra, or JTT matrices as described by Jones et al. (1992) Comput. Appl. Biosci. 8:275-282 and Gonnet et al. (1992) Science 256:1443-1145; the empirical model described by Adach & Hasegawa (1996) J. Mol. Evol. 42:459-468; the block substitution matrices (BLOSUM) as described by Henikoff & Henikoff (1992) Proc. Natl. Acad. Sci. USA 89:1-1; Poisson models as described by Nei (1987) Molecular Evolutionary Genetics. Columbia University Press, New York.; and the Maximum Likelihood (ML) Method as described by Müller et al. (2002) Mol. Biol. Evol. 19:8-13.

Another aspect of the disclosure provides a composition comprising, or alternatively consisting essentially of, or alternatively consisting of, a first polypeptide comprising amino acid residues 11 to 101 of SEQ ID NO. 2, SEQ ID NO. 2 or a substantial homologue or biological equivalent of either one thereof and a second polypeptide selected from a gp41-binding protein, a CCR5-binding protein, a gp120-binding protein or an isolated chimeric polypeptide comprising two different proteins selected from a gp41-binding protein, a CCR5-binding protein, a gp120-binding protein or a substantial homologue or biological equivalent of any one thereof.

In one aspect of the above composition, the chimeric polypeptide is P2-RANTES-linker-C37 or a substantial homologue or biological equivalent thereof.

The mole ratio between the first polypeptide and the second polypeptide can be from about 1:10 to about 10:1, or alternatively from about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1. In another aspect, the mole ratio between the first polypeptide and the second polypeptide is about 1:1.

Another aspect of the composition further comprises a third polypeptide selected from a gp41-binding protein, a CCR5-binding protein, a gp120-binding protein, an isolated chimeric polypeptide comprising two different polypeptides selected from a gp41-binding protein, a CCR5-binding protein or a gp120-binding protein, or a substantial homologue or biological equivalent of any one thereof, the third polypeptide being different from the second polypeptide.

Further provided in the disclosure is a composition comprising, or alternatively consisting essentially of, or alternatively consisting of, a first polypeptide that is at least 80% identical to SEQ ID NO. 2 and a second polypeptide selected from a gp41-binding protein, a CCR5-binding protein, a gp120-binding protein, an isolated chimeric polypeptide comprising two different polypeptides selected from a gp41-binding protein, a CCR5-binding protein or a gp120-binding protein, or a substantial homologue or biological equivalent of any one thereof.

For any of the above composition, the gp41-binding protein can be selected from C37, C34, C52L, T-2635, T20 or a substantial homologue or biological equivalent of any one thereof. In one aspect, the gp41-binding protein is T20 or a substantial homologue or biological equivalent thereof.

For any of the above compositions, the CCR5-binding protein can be selected from RANTES, P2-RANTES, PSC-RANTES, MIP-1α, MIP-1β, U83A, a CCR5 antibody or a substantial homologue or biological equivalent of any one thereof. In one aspect, the CCR5-binding protein is P2-RANTES or a substantial homologue or biological equivalent thereof.

For any of the above compositions, the gp120-binding protein can be cyanovirin-N(CVN), 12p1, CD4M33, CD4M47 or a substantial homologue or biological equivalent of any one thereof.

In another aspect, any of the above compositions further comprises a carrier. The carrier can be a solid phase carrier, a gel, an aqueous liquid carrier, a paste, a liposome, a micelle, albumin, polyethylene glycol, a pharmaceutically acceptable polymer, or a pharmaceutically acceptable carrier, such a phosphate buffered saline.

The compositions of the disclosure can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Compositions may optionally contain stabilizers, pH modifiers, surfactants, bioavailability modifiers and combinations of these.

Compositions may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension compositions may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension compositions.

The compositions of this disclosure are formulated for pharmaceutical administration to a mammal, preferably a human being. Such compositions of the disclosure may be administered in a variety of ways, preferably topically or by injection.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

In addition to dosage forms described above, pharmaceutically acceptable excipients and carriers and dosage forms are generally known to those skilled in the art and are included in the disclosure. It should be understood that a specific dosage and treatment regimen for any particular subject will depend upon a variety of factors, including the activity of the specific antidote employed, the age, body weight, general health, sex and diet, renal and hepatic function of the subject, and the time of administration, rate of excretion, drug combination, judgment of the treating physician or veterinarian and severity of the particular disease being treated.

### Polypeptide Conjugates

Another aspect of the disclosure provides a peptide conjugate comprising, or alternatively consisting essentially of, or alternatively consisting of, a carrier covalently or non-covalently linked to an isolated chimeric polypeptide of the disclosure. In some embodiments, the carrier comprises a liposome, or alternatively a micelle, or alternatively a pharmaceutically acceptable polymer, or a pharmaceutically acceptable carrier.

The polypeptides and polypeptide conjugates of the disclosure can be used in a variety of formulations, which may vary depending on the intended use. For example, one or more can be covalently or non-covalently linked (complexed) to various other molecules, the nature of which may vary depending on the particular purpose. For example, a peptide of the disclosure can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. A peptide can be conjugated to a fatty acid, for introduction into a liposome, see U.S. Pat. No. 5,837,249. A peptide of the disclosure can be complexed covalently or non-covalently with a solid support, a variety of which are known in the art and described herein. An antigenic peptide epitope of the disclosure can be associated with an antigen-presenting matrix such as an MHC complex with or without co-stimulatory molecules.

Examples of protein carriers include, but are not limited to, superantigens, serum albumin, tetanus toxoid, ovalbumin, thyroglobulin, myoglobulin, and immunoglobulin.

Peptide-protein carrier polymers may be formed using conventional cross-linking agents such as carbodimides. Examples of carbodimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

Examples of other suitable cross-linking agents are cyanogen bromide, glutaraldehyde and succinic anhydride. In general, any of a number of homo-bifunctional agents including a homo-bifunctional aldehyde, a homo-bifunctional epoxide, a homo-bifunctional imido-ester, a homo-bifunctional N-hydroxysuccinimide ester, a homo-bifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound may be used. Also included are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homo-bifunctional cross-linking agents include the bifunctional N-hydroxysuccinimide esters dithiobis(succinimidylpropionate), disuccinimidyl suberate, and disuccinimidyl tartrate; the bifunctional imido-esters dimethyl adipimidate, dimethyl pimelimidate, and dimethyl suberimidate; the bifunctional sulfhydryl-reactive crosslinkers 1,4-di-[3′-(2′-pyridyldithio) propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

Examples of common hetero-bifunctional cross-linking agents that may be used to effect the conjugation of proteins to peptides include, but are not limited to, SMCC (succinimidyl-4-(N-maleimidomethyl)cyclohexane-1-carboxylate), MBS (m-maleimidobenzoyl-N-hydroxysuccinimide ester), STAB (N-succinimidyl(4-iodoacteyl)aminobenzoate), SMPB (succinimidyl-4-(p-maleimidophenyl)butyrate), GMBS (N-(γ-maleimidobutyryloxy)succinimide ester), MPBH (4-(4-N-maleimidopohenyl) butyric acid hydrazide), M2C2H (4-(N-maleimidomethyl)cyclohexane-1-carboxyl-hydrazide), SMPT (succinimidyloxycarbonyl-α-methyl-α-(2-pyridyldithio)toluene), and SPDP (N-succinimidyl 3-(2-pyridyldithio)propionate).

Cross-linking may be accomplished by coupling a carbonyl group to an amine group or to a hydrazide group by reductive amination.

The chimeric polypeptides or polypeptides of the compositions of the disclosure also may be formulated as non-covalent attachment of monomers through ionic, adsorptive, or biospecific interactions. Complexes of peptides with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptides may be done to surfaces such as microparticle latex beads or to other hydrophobic polymers, forming non-covalently associated peptide-superantigen complexes effectively mimicking cross-linked or chemically polymerized protein. Finally, peptides may be non-covalently linked through the use of biospecific interactions between other molecules. For instance, utilization of the strong affinity of biotin for proteins such as avidin or streptavidin or their derivatives could be used to form peptide complexes. These biotin-binding proteins contain four binding sites that can interact with biotin in solution or be covalently attached to another molecule. (See Wilchek (1988) Anal. Biochem. 171:1-32). Peptides can be modified to possess biotin groups using common biotinylation reagents such as the N-hydroxysuccinimidyl ester of D-biotin (NHS-biotin) which reacts with available amine groups on the protein. Biotinylated peptides then can be incubated with avidin or streptavidin to create large complexes. The molecular mass of such polymers can be regulated through careful control of the molar ratio of biotinylated peptide to avidin or streptavidin.

Also provided by this application are the peptides and polypeptides described herein conjugated to a label, e.g., a tag (His-tag), label e.g., a fluorescent or bioluminescent label, for use in the diagnostic methods. For example, detectably labeled peptides and polypeptides can be bound to a column and used for the detection and purification of antibodies. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in Haugland, Richard P. (1996) Molecular Probes Handbook.

The chimeric polypeptides or polypeptides of the compositions of the disclosure also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete Adjuvant, Freund's Incomplete Adjuvant and mineral salts.

### Isolated Polynucleotides, Host Cells and Compositions

Yet another aspect of the disclosure provides an isolated polynucleotide encoding for an isolated chimeric polypeptide, an antibody, or a biologically active fragment of the antibody of the disclosure. Also provided is a DNA construct comprising an expression vector and a polynucleotide. In one aspect of the DNA construct, the vector is a plasmid vector, a yeast artificial chromosome, or a viral vector. In one aspect, the vector of the DNA construct comprises a protein tag. Protein tags can be selected from a GST-tag, a myc-tag, or a FLAG-tag provided in expression constructs commercially available from, e.g., Invitrogen, Carlsbad, Calif.

Another aspect of the disclosure provides an isolated host cell transformed with a polynucleotide or a DNA construct of the disclosure. The isolated host cells can be a prokaryotic or a eukaryotic cell. Yet another aspect of the disclosure provides an isolated transformed host cell expressing an isolated chimeric polypeptide, an antibody or a biologically active fragment of the antibody of the disclosure. The isolated host cells can be a prokaryotic or a eukaryotic cell.

Also provided are polynucleotides encoding substantially homologous and biologically equivalent polypeptides to the inventive polypeptides and polypeptide complexes. Substantially homologous and biologically equivalent intends those having varying degrees of homology, such as at least 80%, or alternatively, at least 85%, or alternatively at least 90%, or alternatively, at least 95%, or alternatively at least 98 homologous as defined above and which encode polypeptides having the biological activity as described herein. It should be understood although not always explicitly stated that embodiments to substantially homologous polypeptides and polynucleotides are intended for each aspect of this disclosure, e.g., polypeptides, polynucleotides and antibodies.

The polynucleotides of this disclosure can be replicated using conventional recombinant techniques. Alternatively, the polynucleotides can be replicated using PCR technology. PCR is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) and references cited therein. Yet further, one of skill in the art can use the sequences provided herein and a commercial DNA synthesizer to replicate the DNA. Accordingly, this disclosure also provides a process for obtaining the polynucleotides of this disclosure by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can operatively link the polynucleotides to regulatory sequences for their expression in a host cell. The polynucleotides and regulatory sequences are inserted into the host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

Also provided are host cells comprising one or more of the polypeptides or polynucleotides of this disclosure. In one aspect, the polypeptides are expressed and can be isolated from the host cells. In another aspect, the polypeptides are expressed and secreted. In yet another aspect, the polypeptides are expressed and present on the cell surface (extracellularly). Suitable cells containing the inventive polypeptides include prokaryotic and eukaryotic cells, which include, but are not limited to bacterial cells, algae cells, yeast cells, insect cells, plant cells, animal cells, mammalian cells, murine cells, rat cells, sheep cells, simian cells and human cells. A non-limiting example of algae cells is red alga Griffithsia sp. from which Griffithsin was isolated (Toshiyuki et al. (2005) J. Biol. Chem. 280(10):9345-53). A non-limiting example of plant cells is a Nicotiana benthamiana leaf cell from which Griffithsin can be produced in a large scale (O'Keefe (2009) Proc. Nat. Acad. Sci. USA 106(15):6099-6104). Examples of bacterial cells include Escherichia coli (Giomarelli et al. (2006), supra), Salmonella enteric, Streptococcus gordonii and lactobacillus (Liu et al. (2007) Cellular Microbiology 9:120-130; Rao et al. (2005) PNAS 102:11993-11998; Chang et al. (2003) PNAS 100(20):11672-11677; Liu et al. (2006) Antimicrob. Agents & Chemotherapy 50(10):3250-3259). The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line CHO, BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

### Antibody Compositions

The disclosure, in another aspect, provides an antibody that binds an isolated chimeric polypeptide of the disclosure. The antibody can be a polyclonal antibody, a monoclonal antibody, a chimeric antibody, a humanized antibody or a derivative or fragment thereof as defined below. In one aspect, the antibody is detectably labeled or further comprises a detectable label conjugated to it.

Also provided is a composition comprising the antibody and a carrier. Further provided is a biologically active fragment of the antibody, or a composition comprising the antibody fragment. Suitable carriers are defined supra.

Further provided is an antibody-peptide complex comprising, or alternatively consisting essentially of, or yet alternatively consisting of, the antibody and a polypeptide specifically bound to the antibody. In one aspect, the polypeptide is the chimeric polypeptide against which the antibody is raised.

This disclosure also provides an antibody capable of specifically forming a complex with a protein or polypeptide of this disclosure, which are useful in the therapeutic methods of this disclosure. The term “antibody” includes polyclonal antibodies and monoclonal antibodies, antibody fragments, as well as derivatives thereof (described above). The antibodies include, but are not limited to mouse, rat, and rabbit or human antibodies. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. The antibodies are also useful to identify and purify therapeutic polypeptides.

This disclosure also provides an antibody-peptide complex comprising, or alternatively consisting essentially of, or yet alternatively consisting of, antibodies described above and a polypeptide specifically bound to the antibody. In one aspect the polypeptide is the polypeptide against which the antibody was raised. In one aspect the antibody-peptide complex is an isolated complex. In a further aspect, the antibody of the complex is, but not limited to, a polyclonal antibody, a monoclonal antibody, a humanized antibody or an antibody derivative described herein. Either or both of the antibody or peptide of the antibody-peptide complex can be detectably labeled or further comprises a detectable label conjugated to it. In one aspect, the antibody-peptide complex of the disclosure can be used as a control or reference sample in diagnostic or screening assays.

Polyclonal antibodies of the disclosure can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, which induces the B-lymphocytes to produce IgG immunoglobulins specific for the antigen. This IgG is purified from the mammals serum. Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antiben depot, which allows for a slow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods described in U.S. Pat. Nos. 7,279,559; 7,119,179; 7,060,800; 6,709,659; 6,656,746; 6,322,788; 5,686,073; and 5,670,153.

The monoclonal antibodies of the disclosure can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived therefrom, or any other suitable cell line as known in the art (see, e.g., www.atcc.org, www.lifetech.com., last accessed on Nov. 26, 2007, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present disclosure. The fused cells (hybridomas) or recombinant cells can be isolated using selective culture conditions or other suitable known methods, and cloned by limiting dilution or cell sorting, or other known methods.

In one embodiment, the antibodies described herein can be generated using a Multiple Antigenic Peptide (MAP) system. The MAP system utilizes a peptidyl core of three or seven radially branched lysine residues, on to which the antigen peptides of interest can be built using standard solid-phase chemistry. The lysine core yields the MAP bearing about 4 to 8 copies of the peptide epitope depending on the inner core that generally accounts for less than 10% of total molecular weight. The MAP system does not require a carrier protein for conjugation. The high molar ratio and dense packing of multiple copies of the antigenic epitope in a MAP has been shown to produce strong immunogenic response. This method is described in U.S. Pat. No. 5,229,490 and is herein incorporated by reference in its entirety.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) Bioinvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1997) Microbiol. Immunol. 41:901-907; Sandhu et al. (1996) Crit. Rev. Biotechnol. 16:95-118; Eren et al. (1998) Immunol. 93:154-161 that are capable of producing a repertoire of human antibodies, as known in the art and/or as described herein. Such techniques, include, but are not limited to, ribosome display (Hanes et al. (1997) Proc. Natl. Acad. Sci. USA 94:4937-4942; Hanes et al. (1998) Proc. Natl. Acad. Sci. USA 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass.).; Gray et al. (1995) J. Imm. Meth. 182:155-163; and Kenny et al. (1995) Bio. Technol. 13:787-790); B-cell selection (Steenbakkers et al. (1994) Molec. Biol. Reports 19:125-134.

Antibody derivatives of the present disclosure can also be prepared by delivering a polynucleotide encoding an antibody of this disclosure to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

Antibody derivatives also can be prepared by delivering a polynucleotide of this disclosure to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies of the present disclosure can also be produced using transgenic plants, according to know methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present disclosure can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al. (2000) Infection and Immunity April 68(4):1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits (1998) Advanced Drug Delivery Reviews 31:33-42; Green & Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies of this disclosure also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies of this disclosure can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S, and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Hollinger et al. (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.)

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Fd segments (V_{H}-C_{H}1-VH-C_{H}1) which form a pair of antigen binding regions. Linear antibodies can be bispecific or monospecific.

The antibodies of this disclosure can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present disclosure include naturally purified products, products of chemical synthetic procedures, and products produced by recombinant techniques from a eukaryotic host, including, for example, yeast, higher plant, insect and mammalian cells, or alternatively from a prokaryotic cells as described above.

If a monoclonal antibody being tested binds with protein or polypeptide, then the antibody being tested and the antibodies provided by the hybridomas of this disclosure are equivalent. It also is possible to determine without undue experimentation, whether an antibody has the same specificity as the monoclonal antibody of this disclosure by determining whether the antibody being tested prevents a monoclonal antibody of this disclosure from binding the protein or polypeptide with which the monoclonal antibody is normally reactive. If the antibody being tested competes with the monoclonal antibody of the disclosure as shown by a decrease in binding by the monoclonal antibody of this disclosure, then it is likely that the two antibodies bind to the same or a closely related epitope. Alternatively, one can pre-incubate the monoclonal antibody of this disclosure with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this disclosure.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Methods 74:307.

The isolation of other hybridomas secreting monoclonal antibodies with the specificity of the monoclonal antibodies of the disclosure can also be accomplished by one of ordinary skill in the art by producing anti-idiotypic antibodies. Herlyn et al. (1986) Science 232:100. An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

In some aspects of this disclosure, it will be useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow & Lane (1988) supra.

The antibodies of the disclosure also can be bound to many different carriers. Thus, this disclosure also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding monoclonal antibodies, or will be able to ascertain such, using routine experimentation.

### IV. Methods of the Disclosure

The disclosure, in one aspect, provides a method for preventing or inhibiting HIV entry into a cell, comprising contacting the cell with an effective amount of an isolated chimeric polypeptide or an effective amount of a composition of the disclosure. The cell can be an animal cell, a mammalian cell, or a human cell. In a particular aspect, the cell is a human cell.

Also provided is a method for treating a subject in need thereof, comprising administering to the subject an effective amount of an isolated chimeric polypeptide or an effective amount of a composition of the disclosure. In one aspect, the subject is an HIV patient. In another aspect, the subject is a subject at risk of HIV infection. In one aspect, the subject is an animal, a mammal, or a human. In a particular aspect, the subject is a human.

Route of administration for the methods can be any methods disclosed herein, including but not limited to injection or topical application.

Also provided is a method for preparing an isolated chimeric polypeptide of the disclosure, comprising expressing a polynucleotide encoding the chimeric polypeptide in an isolated prokaryotic or an isolated eukaryotic host cell. Non-limiting examples of host cells include an E. coli cell, lactobacillus, a plant cell, an algae, or a mammalian cell. In one aspect, the method further comprises isolating the polypeptide produced by the isolated host cell.

Accordingly, also provided is an isolated prokaryotic or eukaryotic host cell comprising a polynucleotide of the disclosure, and a composition comprising a carrier and a prokaryotic or eukaryotic host cell as described herein.

The current disclosure, in yet another aspect, provides a method for identifying an agent useful for prevention or treatment of HIV infection, comprising contacting an HIV virus with a cell capable being infected with HIV under suitable conditions, the cell being in contact with a candidate agent and an isolated chimeric polypeptide or a composition of the disclosure, wherein a decrease in infection compared to a cell being in contact with the chimeric polypeptide or the composition only identifies the agent as an agent useful for prevention or treatment of HIV infection.

HIV virus exclusively infects and causes disease in humans therefore so far there are no ideal model exists that can imitate the natural history and pathogenesis of HIV infection and AIDS in the human body. However, the data from animal models provides conceptual insights into immune responses elicited by investigational vaccines, and reassurance of safety, guiding preclinical development and the decision to enter into clinical trials in humans. Non-human primate studies play a leading role in efforts to develop an HIV vaccine.

Macaque monkeys infected with simian immunodeficiency virus (Sly), a virus closely related to HIV can be a good HIV animal model. This model is useful because SIV in macaques follows a similar disease course to HIV. A hybrid virus created by replacing SIV envelope with HIV envelope but retaining the inner core of SIV virus (called SHIVs), replicates acute HIV infection in macaques, and causes rapid disease progression leading to death.

A number of other animal models have been used to obtain information that can have application to HIV. Feline immunodeficiency virus (Fly), transgenic mice that contain part of the HIV genome or co-receptors for viral entry, and severe combined immune deficiency (SCID) mice reconstituted with human immune system cells or tissues are some of the animal models being used to study pathogenesis.

### VI. Kits

An aspect of the disclosure provides a kit for use in preventing or inhibiting HIV entry into a cell, comprising, or alternatively consisting essentially of, or alternatively consisting of, an isolated chimeric polypeptide of the disclosure, and instructions to use.

Also provided is a kit for use in treating a subject in need thereof, comprising, or alternatively consisting essentially of, or alternatively consisting of, an isolated chimeric polypeptide or a composition of the disclosure, and instructions to use.

Kits may further comprise suitable packaging and/or instructions for use of the compositions. The compositions can be in a dry or lyophilized form, in a solution, particularly a sterile solution, or in a gel or cream. The kit may contain a device for administration or for dispensing the compositions, including, but not limited to, syringe, pepitte, transdermal patch and/or microneedle.

The kits may include other therapeutic compounds for use in conjunction with the compounds described herein. These compounds can be provided in a separate form or mixed with the compounds of the present disclosure.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the disclosure, kits for treating a subject who suffers from or is susceptible to the conditions described herein are provided, comprising a container comprising a dosage amount of a composition as disclosed herein, and instructions for use. The container can be any of those known in the art and appropriate for storage and delivery.

Kits may also be provided that contain sufficient dosages of the effective composition or compound to provide effective treatment for a subject for an extended period, such as a week, 2 weeks, 3, weeks, 4 weeks, 6 weeks, or 8 weeks or more.

### EXAMPLES

The disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

A microbicide is a composition that can be used to reduce the infectivity of microbes such as HIV. It can be formulated into a cream or gel and used to prevent sexual spread of HIV. A microbicide can be formulated into a cream or gel and used to prevent sexual spread of HIV. For example, for use in developing countries, the microbicide needs to be inexpensive to produce, stable under high temperature, and active at the lower pH's in the urogenital tract. Some proteins do not have these properties, which is a disadvantage, even though the protein may be very effective in a lab environment. It is known that a single peptide HIV inhibitor, such as T-20/Fuzeon®/enfuvirtide), requires high doses to be effective in human patients, and even though it has an IC₅₀ (50% inhibition) in the quite acceptable 2-20 nM range in many in vitro assays (Root & Steger (2004) Current Pharm. Design 10:1805-25). Griffithsin requires relatively less protein for inhibition in vitro (O'Keefe et al. (2009) PNAS106:6099-6104).

A major drawback of the existing microbicides is that although an inhibitor may work at a very low concentration in vitro, much higher doses are needed to protect a macaque from infection in an in vivo assay (Lederman et al. (2004) Science 306:485-487; Veazey et al. (2005) Nature 438:99-102).

Surprisingly, it has been discovered that the compositions of the current disclosure are even better inhibitors than Griffithsin alone. For instance, in standard cell fusion assays Griffithsin-linker-C37 works better than Griffithsin alone in both R5 and X4 tropic assays as judged by IC₅₀. In addition, when “nonsense” cells are added to provide competition for binding sites (to test whether the compounds have specificity for their targets), Griffithsin-linker-C37 inhibits the cell fusion assay better than Griffithsin alone.

It is known that HIV is able to develop mutations that make it resistant to individual drugs. In the case of compositions of this disclosure, HIV-1 could not easily escape from the chimeric proteins because the compounds utilize two different binding sites.

### Example 1

A diagram of the components of the early steps in the infection process of HIV is shown in FIG. 1. Briefly, the HIV protein gp120 makes contact with cell proteins CD4 and CCR5 (or CXCR4), which leads to exposure of HIV gp41. Part of HIV gp41 enters the membrane of the human cell, and folds onto itself to form a 6 helix bundle, which pulls the viral membrane into proximity of the human cell.

TABLE 1
SEQ ID NO. 1-Polypeptide sequence of Griffithsin:

| | |
|---|---|
| 1 | SLTHRKFGGS GGSPFSGLSS IAVRSGSYLD XIIIDGVHHG GSGGNLSPTF |
| 51 | TFGSGEYISN MTIRSGDYID NISFETNMGR RFGPYGGSGG SANTLSNVKV |
| 101 | IQINGSAGDY LDSLDIYYEQ Y |

TABLE 2
SEQ ID NO. 2-Polypeptide sequence of Griffithsin with an
alanine replacing the unknown amino acid at position 31:

| | |
|---|---|
| 1 | SLTHRKFGGS GGSPFSGLSS IAVRSGSYLD AIIIDGVHHG GSGGNLSPTF |
| 51 | TFGSGEYISN MTIRSGDYID NISFETNMGR RFGPYGGSGG SANTLSNVKV |
| 101 | IQINGSAGDY LDSLDIYYEQ Y |

TABLE 3
SEQ ID NO. 3-Griffithsin-linker-C37:

Start site, Histine tag and fusion cleavage site:

| | |
|---|---|
| 1 | MGGSSHHHHH HSSGLVPR |

Griffithsin:

| | |
|---|---|
| | GS LTHRKFGGSG GSPFSGLSSI AVRSGSYLDA |
| 51 | IIIDGVHHGG SGGNLSPTFT FGSGEYISNM TIRSGDYIDN ISFETNMGRR |
| 101 | FGPYGGSGGS ANTLSNVKVI QINGSAGDYL DSLDIYYEQY |

Linker:

| | |
|---|---|
| | SSSGGGGSGG |
| 151 | GSSSGS |

C37:

HTTW MEWDREINNY TSLIHSLIEE SQNQQEKNEQ ELL

TABLE 4
SEQ ID NO. 4-P2-RANTES-Griffithsin:

Start site, Histine tag and fusion cleavage site:

| | |
|---|---|
| 1 | MGSSHHHHHH SSGLVPRGSH MIEGR |

P2-RANTES:

| | |
|---|---|
| | FSPLS SQSSACCFAY IARPLPRAHI |
| 51 | KEYFYTSGKC SNPAVVFVTR KNRQVCANPE KKWVREYINS LEMS |

Linker:

| | |
|---|---|
| | GGGGSG |
| 101 | GGGS |

Griffithsin:

| | |
|---|---|
| | GSLTHR KFGGSGGSPF SGLSSIAVRS GSYLDAIIID GVHHGGSGGN |
| 151 | LSPTFTFGSG EYISNMTIRS GDYIDNISFE TNMGRRFGPY GGSGGSANTL |
| 201 | SNVKVIQING SAGDYLDSLD IYYEQY |

TABLE 5
SEQ ID NO. 5-Griffithsin-linker-CD4M33:

Start site, Histine tag and fusion cleavage site:

| | |
|---|---|
| 1 | MGGSSHHHHH HSSGLVPR |

Griffithsin:

| | |
|---|---|
| | GS LTHRKFGGSG GSPFSGLSSI AVRSGSYLDA |
| 51 | IIIDGVHHGG SGGNLSPTFT FGSGEYISNM TIRSGDYIDN ISFETNMGRR |
| 101 | FGPYGGSGGS ANTLSNVKVI QINGSAGDYL DSLDIYYEQY |

Linker:

| | |
|---|---|
| | SSSGGGGSGG |
| 151 | GGSSSS |

CD4M33:

CNLH FCQLRCKSLG LLGKCAGSFC ACV

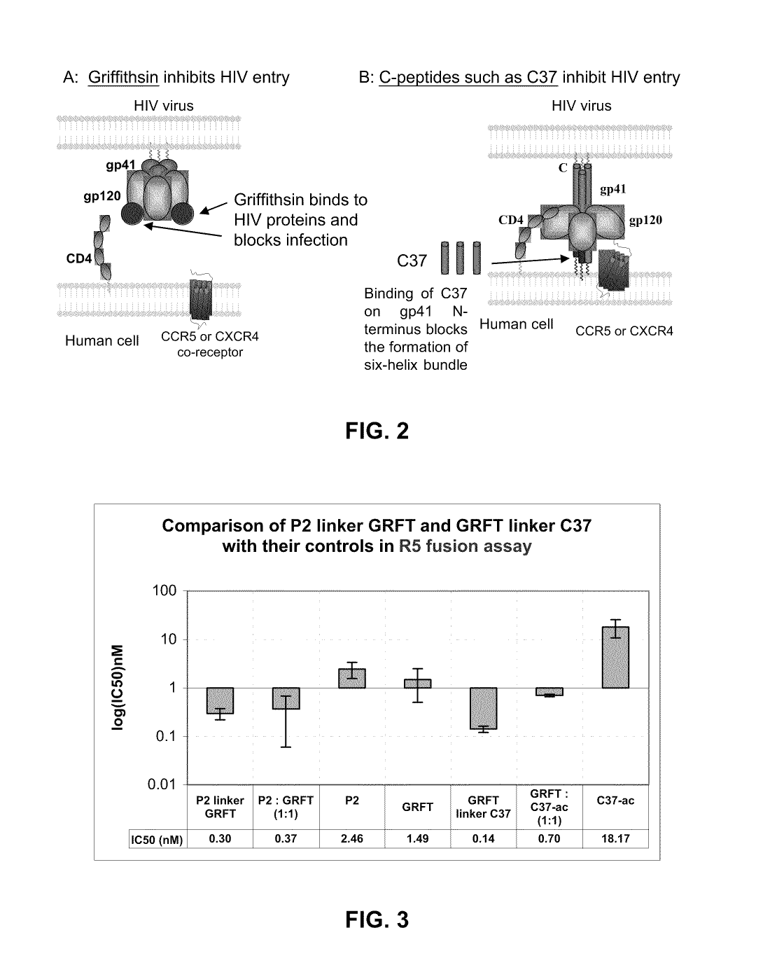

In general, blocking infection by HIV involves stopping one of these processes. For instance, as shown in FIG. 2, Griffithsin binds to sugars on the surface of gp120 and gp41, presumably inhibiting the binding of the virus to the cellular proteins. The peptide C37 (similar to C34 and T-20 and C52L) binds to the N-terminus of HIV gp41, stopping the 6-helix bundle formation.

As shown in FIG. 3, Grft-linker-C37 (SEQ ID NO. 3 in Table 3) was more effective than either Griffithsin alone or C37 alone under several types of assays. In FIG. 3, Grft-l-C37 with Grit was compared to C37 in a cell-cell fusion assay using an R5-tropic effector cell. Also shown in this figure is that P2-RANTES-linker-Griffithsin (as well as P2-RANTES+Griffithsin in combination) were more effective than Griffithsin alone.

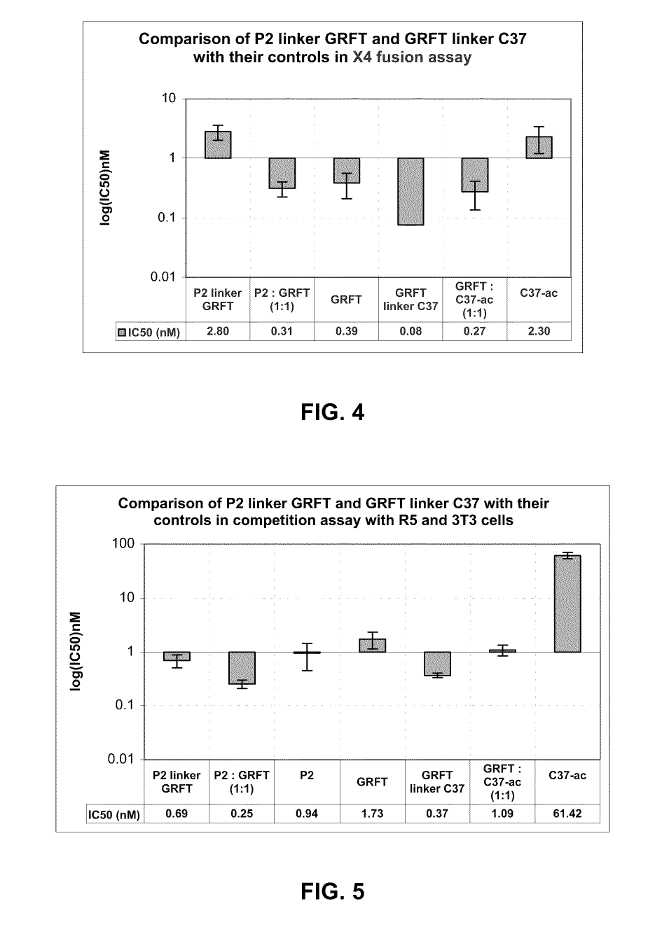

FIG. 4 shows a comparison of Grft-linker-C37 (SEQ ID NO. 3 in Table 3) with Grit and with C37 in a cell-cell fusion assay using an X4-tropic effector cell. As with R5 cells, in X4 tropic assays, Grft-l-037 is a better HIV inhibitor in terms of IC₅₀.

An additional consideration for an HIV inhibitor is whether it will be able to find the correct binding site in a “real life” situation in which there are many different cell types that may provide unproductive binding sites. FIG. 5 shows results of a comparison of Grft-linker-C37 with Grft and with C37 in a cell-cell fusion assay using an R5-tropic effector cell in which there are also competitor cells (mouse 3T3 cells) present in the assay. These competitor cells can not be infected by HIV, but have a milieu of proteins and cell surface sugars that might decrease the effectiveness of an inhibitor and may help the assay provide a reasonable estimation of the ability of an inhibitor to bind to the correct site for inhibition of HIV rather than bind non productively to another cell's surface. As shown in FIG. 5, Grft-linker-C37 unexpectedly performed better than either Griffithsin alone or C37 alone (or the two in unlinked combination). Also, P2-RANTES with Griffithsin (either linked (see, e.g., SEQ ID NO. 4 of Table 4) or unlinked) appeared to perform better than either protein alone, although the unlinked compound appears to be more statistically significant and in general performs better than the linked P2-RANTES-linker-Grft.

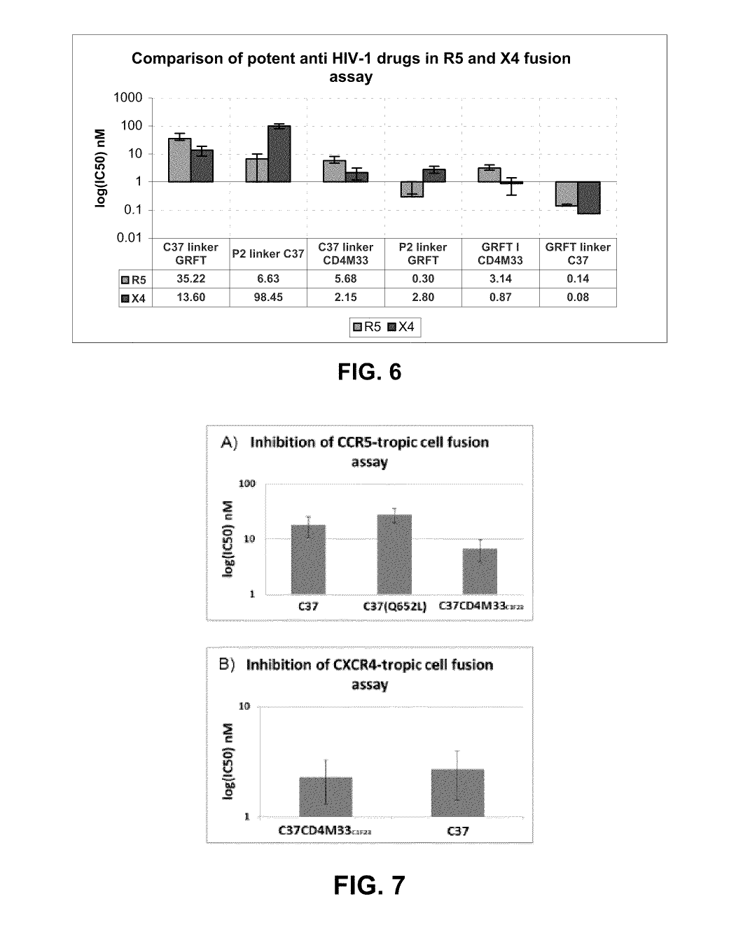

FIG. 6 summarizes some of the results provided above and also shows data for the compound Grft-linker-CD4M33 (SEQ ID NO. 5 of Table 5). The peptide CD4M33 was designed to bind to gp120 in the CD4-binding site, disallowing actual CD4 binding. Overall, Griffithsin-linker-CD4M33 was quite effective in both R5 and X4 cell fusion assays.

### Example 2

The following abbreviations are used in Example 2: Griff37, also referred to as Griffithsin-linker-C37 and also referred to Grft-linker-C37, Griffithsin covalently linked via a 16 amino acid peptide linker with gp41 binding peptide C37; DMEM, Dulbecco's Modified Eagle Medium; HIV, human immunodeficiency virus; TFA, trifluoroacetic acid; CCR5, CC chemokine receptor 5, a co-receptor for HIV entry; PBMC, peripheral blood mononuclear cells; NMR, nuclear magnetic resonance; HSQC, heteronuclear single quantum coherence [spectrum].

This Examples presents evidence that the strategy of linking a gp120 binding molecule with a gp41 binding molecule can lead to a compound that has greater anti-HIV activity than either of its components. It was demonstrated that the highly potent microbicidal candidate Griffithsin could be made even more potent using this strategy.

In the present study, several linked compounds that encompass the strategy of binding both gp120 and gp41 were tested. It was found that one compound, griffithsin-linker-C37 (Griff37) is effective at lower concentrations than griffithsin alone in cell-cell fusion assays and in viral assays, and that this inhibitor exhibits a great deal of specificity under conditions of wash-out and competition. Overall, this and the other HIV inhibitors reported here may be useful as general anti-HIV therapeutics or, more specifically, as anti-HIV microbicides.

### Experimental Methods

### Protein Production and Purification

Peptide fusion inhibitors N-acetylated, C-term amidated C37 and N-acetylated, C-term amidated C37(Q652L) were obtained from Genescript (Piscataway, N.J.). The gene for C37CD4M33_{C1F23} DNA was synthesized by Genscript. All the constructs were placed into pET-15b (Novagen, Madison, Wis.). The amino acid sequence of CD4M33_{C1F23} is: CNLHF CQLRC KSLGL LGKCA GSFCA CV (SEQ ID NO. 6). The amino acid sequence of the linker used for C37CD4M33_{C1F23} is: SSSGG GGSGG GSSSG S (SEQ ID NO. 7) with minor variations between the different constructs due to cloning procedures. The sequence for C37CD4M33_{C1F23} is: MHHHH HHIEG RHTTW MEWDR EINNY TSLIH SLIEE SQNQQ EKNEQ ELLSS SGGGG SGGGG SSSSC NLHFC QLRCK SLGLL GKCAG SFCAC V (SEQ ID NO. 8). The amino acid sequence for C37 is HTTW MEWDREINNY TSLIHSLIEE SQNQQEKNEQ ELL (SEQ ID NO. 9). The amino acid sequence for CD4M33 is CNLH FCQLRCKSLG LLGKCAGSFC ACV (SEQ ID NO. 10). The amino acid sequence for an exemplary histine tag is MGSS HHHHHHSSGL VPRGSH MIEGR (SEQ ID NO. 11). The amino acid sequences for other exemplary linkers are SSSGGGGSGGGSSSGS (SEQ ID NO. 12) and GGGGSGGGGS (SEQ ID NO. 13).

Genes were expressed in BL21(DE3) (Novagen) E. coli cells in LB broth. Protein production was induced upon addition of 1 mM IPTG to a final concentration of 1 mM, followed by incubation for 4 h at 37° C. Pellets from these cells were resuspended in a 30 mL solution (500 mM NaCl, 20 mM Tris (pH 8), 10 mM benzamidine), then French pressed twice at 16,000 psi. After centrifugation for 1 h at 17,000 g, the supernatant was loaded onto a Ni chelating column (Amersham Pharmacia Biotech) equilibrated with 50 mM Tris (pH 8.0), 500 mM NaCl, and eluted with 500 mM imidazole, 50 mM Tris (pH 8.0), 500 mM NaCl. The fractions containing purified protein were dialyzed in a buffer (20 mM Tris (pH 8.0)) at 4° C. overnight. The concentrated protein was further purified on a C4 reversed phase chromatography column (Vydac, Hesperia, Calif.), then lyophilized in a Labconco freeze dry system (Labconco Corporation). For NMR and some functional studies, protein was produced in minimal media with ¹⁵NH₄Cl as the sole nitrogen source, using the same purification procedure after production.

C37CD4M33_{C1F23} was expressed in BL21(DE3) either in LB media or minimal media as indicated above, although the proteins were found in the inclusion body. Therefore, after induction for 4 hours upon addition of 1 mM IPTG at 37° C., the cells were resuspended in 30 mL 5 M guanidinium chloride, 500 mM NaCl, 20 mM Tris (pH 8.0), then French pressed at 16,000 psi. After centrifugation for 1 hour at 17,000 g to remove undissolved material, the supernatant was loaded onto a Ni chelating column (Amersham Pharmacia Biotech) equilibrated with 5M Guanidium, 50 mM Tris (pH 8.0), 500 mM NaCl). Elution was carried out with 5M Guanidium, 500 mM imidazole, 50 mM Tris (pH 8.0), 500 mM NaCl. Fractions containing purified protein were combined and β-mercaptoethanol was added to a final concentration of 10 mM and incubated for 2 h with slow stirring. The protein was then dialyzed in 20 mM Tris (pH 8.0) at 4° C. overnight. The protein was further purified as described above, with C4 reversed phase chromatography followed by lyophilization. For C37CD4M33_{C1F23}, a further step of proteolytic cleavage of the his-tag by Factor Xa was added (reaction buffer: 50 mM Tris-HCl, pH 7.5, 150 mM NaCl, 1 mM CaCl₂). Cleaved protein was finally purified with C4 reversed phase chromatography, and then lyophilized in a Labconco freeze dry system.

The proteins were analyzed by mass spectrometry on an Agilent 1100 HPLC and Thermo Fisher LCQ ion trap mass spectrometer (Stanford University). Expected values based on amino acid sequence are shown in parentheses after each experimental value: C37CD4M33_{C1F23}: 8696 (8709).

### Cell Culture

Six cell lines were used:
1) HeLa-ADA cells that stably expressed HIV-1 ADA (R5) env were maintained in DMEM supplemented with 10% FBS plus 2 μM methotrexate (Sigma) as a selective reagent. This cell line was a kind gift from Dr. M. Alizon (Cochin Institute, Paris, France)).
2) HeLa-P5L cells that stably expressed human receptors CD4 and CCR5 were maintained in RPMI-1640 supplemented with 10% FBS plus 0.5 mg/ml zeocin (Invitrogen) for selection for CCR5 expression. They were a kind gift from Dr. M. Alizon and Dr. Anne Brelot (Cochin Institute, Paris, France).
3) HeLa-TZM-bl cells that stably expressed human receptors CD4, CCR5 and CXCR4 were maintained in DMEM supplemented with 10% FBS. This cell line was obtained through the NIH AIDS Research and Reference Reagent Program, Division of AIDS, NIAID, NIH and was a gift to that program from Dr. John C. Kappes, Dr. Xiaoyun Wu and Tranzyme Inc.
4) HL2/3 cells that stably expressed HXB2 env (X4) was obtained through the NIH AIDS Research and Reference Reagent Program, Division of AIDS, NIAID, NIH and was a gift to that program from Dr. Barbara K. Felber and Dr. George N. Pavlakis. The cells were maintained in DMEM supplemented with 10% FBS and 500 ug/uL G418.
5) 293FT cells were maintained in DMEM supplemented with 10% FBS and were a kind gift from Dr. Jennifer Manilay.
6) Mouse 3T3 cells were maintained in DMEM supplemented with 10% FBS.

### Cell-Cell Fusion Assay

Envelope-medited cell fusion assays have already been described (Pleskoff et al. (1997) Science 276:1874-8). Briefly, for the R5 fusion assay, 10⁴ HeLa-P5L cells (target) per well were seeded in a 96 well plate. After ˜12 hours, media was replaced with 50 μl RPMI-1640. Serial dilutions of inhibitor were added to the wells of the plate: 20 μl of protein or peptide was added to the first well and mixed, then 20 μl was removed and added to the next well and so on. 10⁴ HeLa-ADA cells (effector) in 50 μl RPMI-1640 were then added to each well (100 μl media per well total). The cells were allowed to fuse for 24 hours at 37° C. Cells were then lysed by adding 0.5% NP-40 (US Biological) in PBS for 30 min, then assayed for β-galactosidase activity after addition of 8 mM substrate CRPG (chlorphenol red-β-D-galactopyranoside, Calbiochem) in PBS with 20 mM KCl and 10 mM β-mercaptoethanol (Sigma). The absorbance at 570 nm (signal) and 630 nm (background) was read. The percentage of cell-cell fusion was calculated as [100×(mean absorbance of treated well−mean absorbance of HeLa-P5L-only well)]/(mean absorbance of untreated well−mean absorbance of HeLa-P5L-only well). Kaleidagraph (Synergy Software, Reading Pa.) was used to fit the data into a four-parameter logistic equation.

In the X4 fusion assay, HeLa-TZM cells were used as target cells and HL2/3 cells were used as effector cells.

In the competition R5 fusion assay, 5×10³ HeLa-P5L cells were seeded together with 5×10³ 3T3 cells per well. The rest of the procedure was identical to the normal R5 fusion assay.

In the low temperature R5 fusion assay, the addition of effector cells to target cells was immediately followed by 2 hours of incubation at 16° C. The plate was returned afterwards to the 37° C. cell incubator for 24 hours.

In the wash-out assay, HeLa ADA or HeLa P5L cells were seeded in a 96 well plate the day before the assay. After the formation of the inhibitor gradient, as described above, the cells remained at room temperature for 30 min. Afterwards, the media containing inhibitor was removed and every well was washed with PBS twice. New media without inhibitor was added and the complementary cell (HeLa P5L cells or HeLa ADA cells) were seeded in the plate.

### Single Round Infection Assay

Plasmid pNL-luc3-R⁻E⁻ containing the firefly luciferase gene, pSV-ADA(R5) and pSV-JRFL(R5) were kind gifts from Dr. Nathaniel Landau. For virus production, 293FT cells were double transfected with pNL-luc3-R⁻E⁻ and either pSV-ADA or pSV-JRFL plasmids according to the product manual (ProFection Mammalian Transfection System (Promega)). 48 hours post-transfection, the supernatant was harvested, centrifuged at low speed, and filtered with a 0.45 μm syringe filter. This viral stock was stored at −80° C. For the assay, 10⁴TZM cells per well were seeded in a 96 well plate. The next day, the media was removed and replaced with 50 μl of new media. Serial dilutions of inhibitor were carried out in the wells of the plate: 20 μl of protein or peptide was added to the first well and mixed, then 20 μl was removed and added to the next well and so on. Virus was added in an amount to obtain a luciferase signal between 60,000-80,000 arbitrary units for the ADA-env pseudo virus (while the control of non-infected cells gave ˜600 arbitrary units) and 40,000-60,000 arbitrary units for the JRFL-env pseudo virus. The total volume of media per well after pseudo virus addition was 100 μl. After 24 hours, old media was removed and replaced with new media. A further 24 hours later, the media was removed and the cells were lysed using Glo lysis Buffer (Promega) according to the manual. Luciferase substrate was then added (Luciferase Assay System (Promega)) and the plate was read using an Orion II microplate luminometer (Berthold Techniques, Germany). The percentage of viral infection was calculated as [100×(mean absorbance of treated well−mean absorbance of TZM-only well)]/(mean absorbance of positive control−mean absorbance of TZM-only well). As a positive control the above infection procedure was carried out in the absence of any inhibitor. The results were plotted on Microsoft Excel, and the IC₅₀ and IC₉₀ were calculated using a linear equation fitted between two experimental points surrounding the IC₅₀ or IC₉₀.

### NMR Spectroscopy

Samples that were isotopically labeled with ¹⁵N were prepared by growing BL21(DE3) containing the expression vector pET-15b in the presence of minimal medium containing ¹⁵NH₄Cl as the sole nitrogen source. Samples were dissolved in 20 mM sodium phosphate buffer pH 7.0 with 5% D₂O and a small amount of DSS (2,2-dimethyl-2-silapentane-5-sulfonate) for spectral referencing (Wishart et al. (1995) J. Biomol. NMR 6:135-140). The samples were placed in Shigemi tubes (Allison Park, Pa.). Spectra were measured at 25° C. on a four-channel 600 MHz Bruker Avance III spectrometer equipped with a GRASP II gradient accessory and a TCI cryoprobe, which has an actively-shielded Z-gradient coil and cooled preamplifiers for ¹³C, ¹H, and ²H. ¹H-¹⁵N correlation spectra were measured with 760* points in the ¹H dimension and 64* points in the ¹⁵N dimension, were processed using the program nmrPipe, and visualized using nmrDraw (Delaglio et al. (1995) J. Biomol. NMR 6:277-293).

### Strategically Linked Compounds are Potent in R5-Tropic Cell Fusion Assays.

The potent HIV entry inhibitor, Griffithsin, was covalently linked via a 16 amino acid peptide linker with a gp41 binding peptide C37 to form “Griff37”. Cell fusion assays represent a common method of determining the antiviral potency of many compounds. In this technique, HeLa cells presenting human proteins CD4, CCR5 and/or CXCR4 on their surface are combined with HeLa cells presenting HIV env proteins gp120 and gp41 on their surface. The cells fuse by interaction of their respective surface proteins in an event that generally mimics the HIV-1 infection process. The extent of cell fusion can be measured using a reporter assay, because β-lactamase in the target cell (containing human co-receptors on its surface) is under the control of the LTR promoter, which is activated by Tat from the effector cell “HIV cell” upon fusion [24].

The results of R5 fusion assays are shown in FIG. 7 and Table 6. Griffithsin alone performs well, with an IC₅₀ of 1.31 nM±0.87, and the C-peptide C37 has an IC₅₀ of 18.2±7.6 nM in the R5 assay. The two proteins in combination without being linked inhibit quite well, exhibiting an IC₅₀ of 0.46 nM (for each protein, for a total concentration of 0.91 nM at 50% inhibition). However, when the Griffithsin and C37 are covalently joined by a 16 amino acid linker to form Grft-linker-C37 (hereafter referred to as Griff37), the effectiveness of the compound in the fusion assay increases dramatically, giving an IC₅₀ of 0.15±0.05 nM (FIG. 7A, 7B, Table 6). It has been reported that the activity of C-peptides can be enhanced by making the point mutation Q652L [25], so this point mutation was made in the linked compound. Griff37Q652L was produced and purified, but in an R5 fusion assay this compound has approximately the same effectiveness as Griff37 (Table 6). Conversely, substitution of Asp for Ile at position 642 in C37 has been reported to diminish the activity of C37 and this I642D substitution was made in Griff37, and the resulting protein showed 2.4-fold worse inhibition in the fusion assay than Griff37, but still 2-fold better than griffithsin alone. When the linker between griffithsin and C37 was shortened to 4 amino acids, the resulting protein was 1.6-fold worse than Griff37, but still 3.1-fold better than griffithsin alone.

TABLE 6
Inhibition of HIV cell fusion

| | | | | R5 fusion | R5 fusion |
|---|---|---|---|---|---|
| | | | R5 fusion | with wash- | with wash- |
| | R5 fusion | X4 fusion | with | out on P5L | out on |
| | assay | assay | competition | cells | ADA cells |
| Compound | IC₅₀ nM | IC₅₀ nM | IC₅₀ nM | IC₅₀ nM | IC₅₀ nM |
| Griffithsin | 1.305 ± 0.865 | 0.468 ± 0.265 | 1.732 ± 0.603 | 7.341 ± 3.583 | 4.28 ± 1.497 |
| Griff37 | 0.148 ± 0.052 | 0.088 ± 0.033 | 0.369 ± 0.034 | 1.409 ± 0.750 | 3.394 ± 0.556 |
| | (0.0) | (0.43) | (0.9) | (0.) | (0.3933) |
| GriffC37(Q652L) | 0.121 ± 0.056 | 0.167 ± 0.085 | N.D.⁵ | N.D.⁵ | N.D.⁵ |
| | (0.3) | (0.) | | | |
| Griffithsin + | 2.013 ± 0.659⁴ | 1.348 ± 0.650 | N.D.⁵ | N.D.⁵ | N.D.⁵ |
| C37(Q652L) (1:1) | | | | | |
| C37CD4M33_{C1F23}² | 6.835 ± 2.850 | 2.299 ± 0.982 | 7.780 ± 1.563 | 560.71 ± 317.87 | 77.795 ± 1.175 |
| GriffCD4M33_{C1F23}² | 3.471 ± 0.966 | 1.186 ± 0.443 | 12.555 ± 1.405³ | 50.705 ± 8.975 | |
| C37¹ | 18.168 ± 7.457 | 2.696 ± 1.266 | 61.420 ± 7.884 | No inhibition | No inhibition |
| C37(Q652L)¹ | 28.160 ± 8.340 | N.D.⁵ | N.D.⁵ | N.D.⁵ | N.D.⁵ |
| C37-uncapped¹ | >100 | N.D.⁵ | N.D.⁵ | N.D.⁵ | N.D.⁵ |

IC₅₀ values for fusion assays under various conditions. Each experiment was done in triplicate and repeated at least 3 times except where noted. Numbers in parentheses indicate the p value resulting from a T test of that compound in comparison to Griffithsin.
¹The peptide C37 is a product of chemical synthesis and is capped at both ends, with N-terminal acetylation and C-terminal amidation. “C37-uncapped” contains native N-and C-termini, which leads to less effective inhibition.
²CD4M33_{C1F23} is a recombinantly produced protein based upon the synthetically produced peptide CD4M33 designed by Martin et al (REF). When not in combination in the studies, the peptide also has a His-tag attached because the peptide could not be successfully cleaved without excessive loss. When in combination with C37, the His tag of the linked compound was removed.
³This experiment was done in triplicate repeated two times.
⁴Total protein concentration.
⁵Not determined.

The peptide C37 was linked with a 16 amino acid linker to a modified version of the peptide CD4M33, an HIV inhibitor that was designed to bind gp120 in a manner similar to the protein CD4 (Martin et al. (2001) Biochemistry 40:6303-18). While the published peptide contains unnatural amino acids in positions 1 and 23, these amino acids were replaced with natural amino acids Cys and Phe respectively in order to allow expression in E. coli. This peptide was referred to as CD4M33_{C1F23}. In CCR5-tropic fusion assays, C37-linker-CD4M33_{C1F23} (C37CD4M33_{C1F23}) exhibited an IC₅₀ of 6.84±2.9 nM (FIG. 7 and Table 6). This value is significantly lower than that for either component alone in cell-cell fusion assays, since C37 has an IC₅₀ of 18.2 nM, and his-tag-containing CD4M33_{C1F23} was not an effective inhibitor even at the highest tested amount of 1.4 uM. Published reports of CD4M33 also describe that micromolar amounts of the peptide are required to inhibit CCR5-tropic fusion in a cell-cell fusion assay using vaccinia technology (Martin et al. (2001) Biochemistry 40:6303-18). Applicants relied on published reports of the activity of unlinked CD4M33, because although Applicants were able to recombinantly make CD4M33 linked proteins, they were unable to successfully produce in E. coli or synthesize enough of the individual peptide to test at the micromolar concentrations that are required for inhibition in fusion assays.

The compound Griffithsin-linker-CD4M33_{C1F23}, which could provide two molecules to bind to gp120, was also produced. This compound performed reasonably well in R5 cell fusion assays, with an IC₅₀ of 3.47±0.97 nM.

These results suggest the possibility that combining a gp120 binding protein (such as Griffithsin) with a gp41 binding protein (such as C37) is a potent strategy for R5HIV inhibition.

The R5 fusion assays reported above do not involve pre-incubation of the inhibitors, but rather the direct addition of inhibitor at approximately the same time as the effector cell. To address the possibility that pre-incubation may enhance the effectiveness of the inhibitors, an altered protocol was used. In this experiment, both target and effector cells were present on the plate at lowered temperature (16° C.), so that fusion could not occur [27], and inhibitor was added. The cells and inhibitor were kept at 16° C. for 2 hours before the temperature was raised to 37° C. to allow fusion to proceed. Under these conditions, the inhibitors performed about the same in terms of IC₅₀ as in a regular fusion assay.

### Strategically Linked Compounds are Potent in X4-Tropic Cell Fusion Assays.

The linked combination of Griffithsin and C37 was also quite potent in X4-tropic cell fusion assays. As shown in FIG. 7C, 7D and Table 6, while Griffithsin alone exhibited an IC₅₀ of 0.48±0.26 nM, the linked Griff37 was 5.2 fold more potent, similar to the point mutant of the linked compound, Griff37Q652L (Table 6). When unlinked, the combination of Griffithsin and C37 were only slightly more potent than each separately (FIG. 7 and Table 6). For these assays, it was found that although C-peptide inhibitors are sensitive to the condition of the assays, Griffithsin and Griff37 give quite consistent results over a wide range of conditions such as cell age or cell type.

When C37 was linked with the gp120-binding peptide CD4M33_{C1F23}, the resulting C37CD4M33_{C1F23} gave an IC₅₀ of 2.30±0.98 nM. Since this is close to the value of C37 alone, and CD4M33 has been shown to inhibit in an X4 fusion assay in the micromolar range (IC₅₀ of 0.8±0.09 uM) (Martin et al. (2003) Nat. Biotechnol. 21:71-6), it appears that linking the two inhibitors in this case did not improve inhibition. Interestingly, these results differ from the R5 cell fusion assay, where the C37CD4M33_{C1F23} compound gave an IC₅₀ that was significantly better than either compound alone (FIG. 7 and Table 6).

### Strategically Linked Compounds Perform Well in Viral and Pseudoviral Assays.

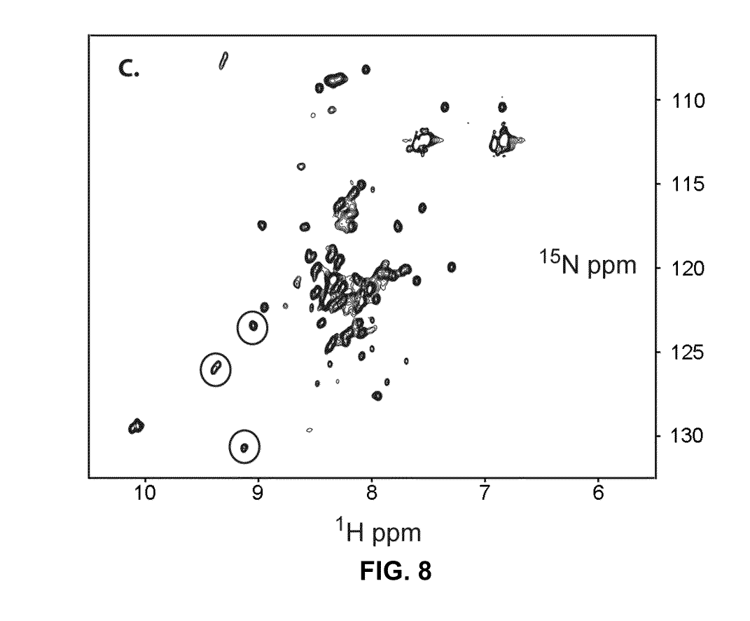

To confirm that the strategy of linking a gp120-binding protein with a gp41-binding peptide is successful in inhibiting HIV virus, a series of assays were performed, using several types of target cells and viral strains. In replication competent HIV assays, the linked compound Griff37 consistently performs better than Griffithsin alone. For example, when the R5 strain Ba-L is used to infect MAGI cells, Griffithsin alone exhibits an IC₅₀ of 0.04 nM±0.01, while Griff37 is 2.7 fold better, with an IC₅₀ of 0.015±0.005 nM (Table 7, FIG. 8) and the unlinked combination of Griffithsin and C37 has an IC₅₀ of 0.05 nM. Similarly, when the R5 strain ADA infects MAGI cells, Griff37 is 2 fold better than Griffithsin alone and 6.3 fold better than the unlinked combination of Griffithsin and C37 (Table 7). The effect is even more dramatic when comparing concentration at 90% inhibition (IC₉₀'s) for these compounds against viral strains: For BaL, Griff37 is 6.4 fold more potent than Griffithsin and 9.4 fold more effective than the unlinked combination (Table 7); similar results are observed for inhibition of strain ADA.

TABLE 7
Inhibition of HIV virus

| | Ba-L (R5) in | | ADA (R5) in | | IIIB (X4) in MAGI | |
|---|---|---|---|---|---|---|
| | MAGI cells | | MAGI cells | | cells | |
| Compound | IC₅₀ nM | IC₉₀ nM | IC₅₀ nM | IC₉₀ nM | IC₅₀ nM | IC₉₀ nM |
| Griffithsin | 0.040 ± 0.010 | 0.515 ± 0.055 | 0.030 ± 0.010 | 0.450 ± 0.040 | 0.145 ± 0.065 | 0.890 ± 0.110 |
| C37¹ | 7.560 ± 1.070 | 79.650 ± 2.450 | 6.145 ± 1.395 | 68.850 ± 4.150 | 10.250 ± 1.850 | >100 |
| Griffithsin + | 0.100 ± 0.030 | 0.750 ± 0.020 | 0.095 ± 0.015 | 0.705 ± 0.005 | 0.230 ± 0.020 | 2.210 ± 0.570 |
| C37 | | | | | | |
| GriffC37 | 0.015 ± 0.005 | 0.080 ± 0.010 | 0.015 ± 0.005 | 0.085 ± 0.005 | 0.035 ± 0.005 | 0.325 ± 0.065 |
| | (0.1548) | (0.0161) | (0.3118) | (0.0120) | (0.2336) | (0.0475) |

| | Ba-L (R5) in | | 91US005 (R5) in | |
|---|---|---|---|---|
| | PBMC | | PBMC | |
| Compound | IC₅₀ nM | IC₉₀ nM | IC₅₀ nM | IC₉₀ nM |
| Griffithsin | 0.280 ± 0.170 | 0.760 ± 0.150 | 0.160 ± 0.030 | 0.510 ± 0.080 |
| C37¹ | 2.385 ± 0.635 | 7.400 ± 1.200 | 29.525 ± 28.475 | 70.750 ± 19.550 |
| Griffithsin + | 0.165 ± 0.025 | 0.485 ± 0.165 | 0.205 ± 0.035 | 0.805 ± 0.075 |
| C37 | | | | |
| GriffC37 | 0.059 ± 0.0004 | 0.155 ± 0.065 | 0.085 ± 0.005 | 0.430 ± 0.150 |
| | (0.3087) | (0.0659) | (0.1325) | (0.6842) |

IC₅₀ values for viral assays. Numbers in parentheses indicate the p value resulting from a T test of that compound in comparison to Griffithsin. Each experiment was done in triplicate and repeated 2 times.
¹The peptide C37 is a product of chemical synthesis and is capped at both ends, with N-terminal acetylation and C-terminal amidation. C37-uncapped contains native N-and C-termini, which leads to less effective inhibition.

As described for fusion assays, these compounds are also highly effective against X4 strains. When MAGI cells are infected by the X4 strain IIIB, Griff37 again shows several fold higher potency than its components (Table 7).

Similar results are observed when human PBMC are infected with Ba-L. In this case, griffithsin alone has an IC₅₀ of 0.28±0.17 nM, while Griff37 is 5.6 fold better, with an IC₅₀ of 0.059±0.0004 nM, and the unlinked components griffithsin plus C37 are less than two-fold better than griffithsin alone (Table 7). When the R5 primary strain 91 US005 is used to infect PBMC, the result is about a two fold improvement for Griff37 over griffithsin alone (Table 7). GriffC37 also shows a 3.8-fold increase in anti-HIV potency compared to Griffithsin alone in CXCR4-tropic strains NL4-3 and 92HT599 (primary strain; Table 8). Since the griffithsin-containing compounds were clearly more potent than the CD4M33_{C1F23}-containing compounds, the CD4M33_{C1F23} compounds were not tested in replication competent viral assays.

TABLE 8
Inhibition of HIV-1 Replication in PBMCs

| | Ba-L | | 91US005 | | NL4-3 | | 92HT599 | |
|---|---|---|---|---|---|---|---|---|
| | (CCR5-tropic) | | (CCR5-tropic) | | (CXCR4-tropic) | | (CXCR4-tropic) | |
| Compound | IC₅₀ nM | IC₉₀ nM | IC₅₀ nM | IC₉₀ nM | IC₅₀ nM | IC₉₀ nM | IC₅₀ nM | IC₉₀ nM |
| Griffithsin | 0.280 ± 0.17 | 0.760 ± 0.15 | 0.280 ± 0.17 | 0.760 ± 0.15 | 0.170 ± 0.04 | 0.725 ± 0.13 | 0.74 ± 0.37 | 1.86 ± 0.89 |
| C37 | 2.39 ± 0.64 | 7.40 ± 1.2 | 2.39 ± 0.64 | 7.40 ± 1.2 | 289 ± 0.39 | 8.33 ± 0.25 | 14.5 ± 1.4 | 27.2 ± 0.60 |
| Griffithsin + | 0.33 ± 0.05 | 0.97 ± 0.33 | 0.33 ± 0.05 | 0.97 ± 0.33 | 0.220 ± 0.04 | 0.58 ± 0.02 | 0.76 ± 0.24 | 1.77 ± 0.03 |
| C37 (1:1) | | | | | | | | |
| Griff37 | 0.059 ± 0.0004 | 0.155 ± 0.07 | 0.059 ± 0.0004 | 0.155 ± 0.07 | 0.045 ± 0.005 | 0.205 ± 0.04 | 0.195 ± 0.03 | 0.630 ± 0.07 |
| | (0.044) | (0.20) | (0.077) | (0.16) | (0.049) | (0.011) | (0.14) | (0.004) |

IC50 and IC90 values for viral replication assays. All combinations are reported as total protein concentration, and a 1:1 ratio indicates equal molar amounts. Numbers in parentheses indicate the p-value resulting from a t-Test of that compound in comparison to Griffithsin + C37. A value of p < 0.05 is considered statistically significant. Each experiment was done in triplicate and repeated 2 times. The IC50 and IC90 values were calculated as the averages of independent experiments ± the Standard Deviation.

In single-round infection assays Griffithsin and its analogs all perform quite well. Virions pseudotyped with the R5JRFL strain and R5ADA of HIV were both used in inhibition assays. While Griff37 performed better than Griffithsin alone with strain JRFL, it did not perform better using strain ADA (Table 9). Indeed, using strain ADA in a single round infection assay was the only instance identified in which Griff37 was not superior to Griffithsin (Tables 6, 7, 9). In addition, the combination of gp41 binding peptide C37 with gp120-binding CD4M33_{C1F23}, C37CD4M33_{C1F23}, also appears to perform better than the components CD4M33 or C37 alone in R5 pseudoviral assays (Table 9 and (17)).

TABLE 9
Inhibition of HIV single round pseudovirus in TZM-bl cells

| | ADA (R5) | | JRFL (R5) | |
|---|---|---|---|---|
| Compound | IC₅₀ nM | IC₉₀ nM | IC₅₀ nM | IC₉₀ nM |
| Griffithsin | 0.008 ± 0.016 | 0.164 ± 0.069 | 0.035 ± 0.008 | 1.252 ± 0.190 |
| Griff37 | 0.020 ± 0.016 | 0.172 ± 0.061 | 0.025 ± 0.022 | 0.260 ± 0.101 |
| | (0.043) | (0.779) | (0.377) | |
| Griffithsin + C37 (1:1) | 0.021 ± 0.018 | 0.277 ± 0.184 | 0.032 ± 0.017 | 1.379 ± 0.393 |
| GriffC37(Q652L) | 0.023 ± 0.006 | 0.274 ± 0.066 | N.D. | N.D. |
| | (0.004) | (0.052) | | |
| Griffithsin + | 0.021 ± 0.006 | 0.374 ± 0.192 | N.D. | N.D. |
| C37(Q652L) (1:1) | | | | |
| C37CD4M33_{C1F23}² | 2.332 ± 2.831 | 17.288 ± 17.422 | 2.251 ± 1.391 | 24.316 ± 19.577 |
| GriffCD4M33_{C1F23}² | 0.222 ± 0.017 | 2.693 ± 0.226 | 0.270 ± 0.147 | 2.889 ± 0.254 |
| C37¹ | 27.187 ± 17.166 | 636.643 ± 290.118 | 15.162 ± 4.208 | 462.45 ± 356.62 |
| C37(Q652L)¹ | 114.370 ± 4.939 | N.D. | N.D. | N.D. |

IC₅₀ and IC₉₀ values for single round pseudovirus assays. Numbers in parentheses indicate the p value resulting from a T test of that compound in comparison to Griffithsin. Each experiment was done in triplicate and repeated at least 3 times.
¹The peptide C37 is a product of chemical synthesis and is capped at both ends, with N-terminal acetylation and C-terminal amidation. C37-uncapped contains native N-and C-termini, which leads to less effective inhibition.
²N.D.: Not Determined

### Strategically Linked Compounds Maintain Effectiveness in Competition Assays and in Washing Assays.

For a potential therapeutic to be beneficial in an organism, it needs to be able to effectively find its target in the milieu of other cell types and potential binding partners. As a test of this ability, the R5 cell fusion assay was modified with the addition of unrelated mouse 3T3 cells. These cells are not able to be infected by HIV and do not have human coreceptors on their surface, but they present a myriad of proteins and carbohydrates that could potentially bind an anti-HIV therapeutic and confound its ability to inhibit in the assay.

As previously reported, in an R5 fusion assay in the presence of unrelated competitor cells, the peptide C37 performs significantly worse than in the absence of such cells, with an IC₅₀ of 61.4±7.9 nM ([28] and Table 6). This leads to the possibility that Griff37 also performs worse in the presence of unrelated cells due to the presence of C37. However, the linked compounds perform quite well under these conditions. The presence of competitor cells does not greatly affect griffithsin-containing compounds, as griffithsin alone still performs well. But Griff37 again performs significantly better than griffithsin alone (Table 6). When C37 is linked to gp120-binding peptide CD4M33 to make C37CD4M33_{C1F23}, the resulting IC₅₀ is 7.78±1.6, which is close to the value in the absence of competition. Similar results were also observed for Griff37 in single round infection assays using virus pseudotyped with the R5 strain JRFL in the presence of competitor cells. In these experiments, all griffithsin-containing assays performed well, but Griff37 performed better than griffithsin alone (Table 7).

To determine whether the linked inhibitors maintain some of their activity under conditions of washing out, modified R5 fusion assays were carried out. In these assays, either the target cell or effector cell (“HIV cell”) were placed in a well in the presence of inhibitor. The supernatant was then removed and replaced by inhibitor-free serum, followed by the addition of the other cell type to allow fusion to proceed. Therefore, inhibition would only be observed for inhibitors that can maintain their presence rather than be washed away. In these assays, an IC₅₀ for C37 could not be determined because it was apparently fully washed out. However, both griffithsin and Griff37 maintained nanomolar effectiveness when first bound to the ADA “HIV cell” before the washing step. When the inhibitors were incubated with the target cell before the washing step, both inhibitors still performed well, but Griff37 was 5.2 more effective than griffithsin alone (Table 6). Overall these experiments indicate that under physiological conditions where many different cell types and fluids may be present, these inhibitors maintain very high effectiveness.

### NMR Experiments on the Anti-HIV Compounds

Nuclear magnetic resonance (NMR) is a very effective technique for determining structural details of proteins, including their extent of foldedness and the possibilities for their oligomeric state [29-31]. FIG. 8A shows the ¹⁵N-¹H correlation spectrum (¹⁵N HSQC) of Griffithsin alone, which is a 121 amino acid protein that crystallizes as a dimer [15]. This type of spectrum shows one peak for every N—H pair in the protein, and therefore provides a fingerprint that is unique for each protein. The spectrum of griffithsin shows good peak dispersion in the ¹H dimension with well-resolved peaks, strongly suggesting a nicely folded protein, as would be expected for a stable, wild type, relatively low molecular weight protein.

FIG. 8B shows the spectrum of the linked compound Griff37. Despite this protein exhibiting potent anti-HIV activity in numerous assays, the spectrum shows strong signal around 8.2 ppm in the ¹H dimension, indicative of unfolded or random coil protein in the sample. There are also peaks (of lower intensity) in the “folded” region of the spectrum (above 9 ppm), and an overlay of this spectrum with griffithsin alone indicates that these peaks arise from folded griffithsin (overlay not shown). There are several likely explanations for the poor quality of this spectrum despite the protein being quite active. First, the 16 amino acid linker between Griffithsin and C37 was designed to be structurally flexible, so the signal from this region is expected to resonate in the “unfolded” region of the spectrum. Second, the peptide C37 is likely unfolded in the absence of a binding partner [32], which would lead to peaks in the unfolded region of the spectrum due to this peptide, even if the peptide would be fully active when presented with a binding partner in an assay. Finally, if the Griff37 protein forms oligomers or loose aggregates, line broadening would occur, which would have the effect of decreasing the intensity of all the peaks, particularly those from residues that are not free to move quickly in solution. Therefore, a globular protein like griffithsin would be expected to lose a great deal of signal intensity upon oligomerization. Overall, the spectrum of Griff37 suggests a sample that is at least partially folded (due to the clear presence of peaks that overlap with those in the griffithsin sample) but that may have a percentage of the sample unfolded and/or a portion of the sample in an oligomerized state.

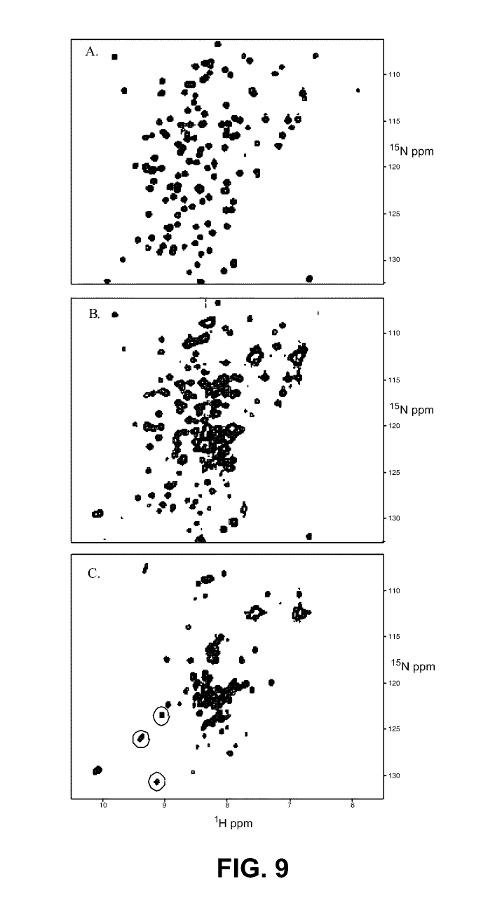

NMR experiments were also carried out on the C37CD4M33_{C1F23} peptide combination. Since this compound is a fusion of two peptides (albeit functionally active peptides), the unfolded peaks that are observed in the 8.2 ppm region of FIG. 9 were expected. However, there were also peaks indicative of folded protein (circled in FIG. 9). Since C34 (nearly identical to C37) has been shown to be unfolded in the absence of its gp41 binding partner (Lu & Kim (1997) J. Biomol. Struct. Dyn. 15:465-71), it is likely that the peaks in this region of the spectrum result from folded CD4M33_{C1F23}. The parent compound, CD4M33, was designed by Martin et al to be a folded peptide containing unnatural amino acids and was produced for their work by chemical synthesis (Martin et al. (2003) Nat. Biotechnol. 21:71-6). Therefore, it is notable that although multiple changes were made to CD4M33, including the use of “natural” amino acids to replace the designed unnatural amino acids, the use of recombinant expression rather than synthesis, and linkage with another peptide, the resulting protein still is an active anti-HIV compound that is at least partly folded.

Many binding targets, both on the HIV virion and on the human cell, have been established as effective sites for HIV inhibition. But an underexplored area for HIV inhibition is to elucidate an overall strategy, possibly combining multiple inhibitors, that optimizes the effectiveness of inhibition under a number of different conditions and viral strains. The present work examines the possible benefits of combining in a single compound both a gp120-binding and a gp41-binding moiety. It was found that such a strategy consistently performs well both in fusion assays and in viral assays, in both R5 and X4 strains, as well as under conditions of competition and washing out. In most cases, the linked compounds performed better than their parent compounds, most strikingly in the case of improvement of the already highly potent protein griffithsin.

Griffithsin has shown a great deal of promise in tests of its anti-HIV microbicidal characteristics. In addition to remarkably high potency, it is stable upon incubation at 37° C., which is a necessary property if it must remain active in the human body for hours or be stored without refrigeration [13]. It also retains its activity in cervical/vaginal lavage fluid [13], is non-inflammatory in human cervical explants, is non irritating in a rabbit vaginal model and is active against multiple clades of HIV, indicating likely usefulness in many or all of the areas hardest hit by this disease [14].

The most effective inhibitor in the experiments was Griff37 or possibly its slight variant, Griff37Q652L, both compounds that covalently link griffithsin with the C-peptide C37. This linked inhibitor performed at sub-nanomolar levels in both R5 and X4 fusion assays, and at mid-picomolar levels in viral assays. These values are in almost every case better than for griffithsin alone and better than an unlinked combination of griffithsin and C37 (Tables 6, 7, 8). This indicates that the linked combination of inhibitors is indeed more potent that the individual components and worthy of strong consideration as a microbicide or therapeutic. In the replication competent viral assays, the improvement in the IC₉₀ for the linked compound Griff37 was more statistically significant than the IC₅₀, which is a positive indicator since 90% inhibition is perhaps more critical for truly stopping infection. In part, this variation in statistical significance is due to those assays being performed fewer times than the others reported. However, the effectiveness of Griff37 at the higher concentrations needed for 90% inhibition could be partly explained by the increasing importance of the C37 component at these concentrations. As the concentration of the inhibitor rises from picomolar levels (where griffithsin binds to gp120), to nanomolar levels, the C-peptide may contribute more to the overall inhibition. As a further test of the strategy of covalently linking a gp120-binding compound with a gp41-binding compound, the linked peptide C37CD4M33_{C1F23} was produced. Again, this compound consistently showed more potent inhibition than either component separately in the R5 cell fusion assay (Tables 6 and 8).

In deriving a set of possible models for the explanation of the effectiveness of Grifff37, each model must account for the fact that this linked molecule is a significantly better inhibitor than the unlinked combination of the two components. In keeping with this, there are at least three plausible models for the mechanism of action of Griff37. The first model is that the griffithsin moiety binds to gp120 with high affinity, and the role of the linked C37 is simply to provide a more sizeable protein on gp120, making a more effective blockade against binding to CD4 or CCR5. However, arguing against this simple steric mechanism is the evidence that griffithsin linked to the peptide CD4M33 (Griff CD4M33_{C1F23}) also would be expected to be able to provide a similar binding blockade, and yet this molecule does not show enhanced inhibition compared to griffithsin alone. Also, Griff371642D, which carries a substitution (substitution of Asp for Ile at position 642 in C37) that negatively affects C37 binding to gp41, shows decreased activity. This indicates that C37 function is important to the activity of the linked compound. In one aspect, the term “biological equivalent”, with respect to C37, excludes the wild type C37 and those having at least 70, 75, 80, 85, 90, 95 or 98 percent identity which contain the Asp substitution for Ile at position 642. Stated another way, the term “biological equivalents” does not include those having at least 70, 75, 80, 85, 90, 95 or 98 percent identity but that amino acid at position 642 is altered from Ile. A second model is one in which the griffithsin component binds to gp120, thereby delivering C37 close to its binding site on gp41. By being in physical proximity to gp41, C37 is able to bind gp41 and inhibit fusion if and when gp41 is exposed, possibly dislodging the linked griffithsin at that time. In this scenario, it is assumed that only one component of the linked pair is binding to its site at any given time, although they may both be in an on-off equilibrium with their respective binding sites.

In the third model of action for Griff 37, both components (i.e. griffithsin and C37) are bound to their respective targets simultaneously. The 16 amino acid linker used in these inhibitors was designed to be flexible and so to allow simultaneous binding of the two components even if the binding sites were fairly distant. For example, an 8 amino acid extended chain in griffithsin spans about 24 Å, so a 16 amino acid linker could potentially allow binding at sites several tens of angstroms apart. In the structure and structural model provided by Chen et al. in their work on unliganded SIV gp120, much of the surface of gp120 is close enough to gp41 to allow simultaneous binding of griffithsin and C37. However, the presence of griffithsin is very likely to stop the binding of gp120 to CD4, and it has been shown that structural changes in gp120 to allow exposure of gp41 does not occur unless CD4 are bound. Therefore, a “simultaneous binding” model of Griff37 action could possibly include two scenarios. In one scenario, griffithsin is bound to carbohydrate on gp120, but not in a correct position to abrogate CD4 binding, which allows gp120 to contact CD4 and expose gp41. The linked C37 moiety of Griff37 can bind gp41 and in this case provides “rescue” for the ineffective inhibition of griffithsin. Alternatively, the griffithsin of Griff37 may inhibit CD4 binding on the monomer of gp120 to which it is bound, but a neighboring gp120 of the trimer may contact CD4 and alter its conformation enough to expose the gp41 trimer. In this case, both griffithsin and C37 in the linked molecule can effectively bind to their target sites.

Similarly, experiments reported here with linked peptides also indicates the beneficial effects of binding both gp120 and gp41, although E. coli production without optimization leads to the possibility that the effect could be stronger after improved purification conditions are determined. The compound C37CD4M33_{C1F23} inhibits in R5 fusion assays much better than either component alone, indicating the effectiveness of binding both targets of these compounds, possibly at the same time. Small differences in IC₅₀ values between C37 and C37CD4M33_{C1F23} in the X4 cell fusion assay may be explained by a lower affinity of CD4M33_{C1F23} for HXB2 env. The lack of a proper control in this case (CD4M33_{c1F23} alone) is critical. It should be noted that the values for this version of CD4M33_{C1F23} are expected to be significantly worse than the reported values for the CD4M33 peptide, since ours is fully recombinant without the benefit of selectively designed unnatural components. Another important point is the high number of cysteines (6 total) that C37CD4M33_{C1f23} has that could allow for potentially different combinations of S—S bonds. Even if C37CD4M33_{C1F23} shows several folded peaks in the NMR spectrum, it is not clear that any or all of the proper disulfide bonds are formed. Therefore, it is possible that CD4M33_{C1F23} does not have the proper folding to bind as tightly to gp120 as the chemically synthesized CD4M33. Even with these drawbacks, it is fairly easy and economical to produce C37CD4M33_{C1F23}, and its relatively small size makes it an attractive candidate for further improvement.

It is possible that there are additional benefits to the linked compounds aside from improved potency. The C-peptide T-20 is clinically approved but suffers from the need for frequent dosing due to a short lifetime of only hours in vivo. It has been shown that linking the similar peptide C34 with albumin (a larger protein) allows for high detectable plasma concentrations of the linked compound in rats for days, suggesting that linking a C-peptide with a larger protein may delay renal clearance and/or susceptibility to protease degradation. These experiments involve the protein C37, which, like T-20, is derived from the sequence of gp41 and which is identical to C34 but has an additional 3 amino acids on the N-terminus. It is possible that Griff37 has an in vivo lifetime of several days due to its larger size, similar to albumin linked C34. In addition, a benefit of a drug with two different sites of action is the difficulty of viral escape, since viral mutations to escape from one of the linked inhibitors would be expected to have little or no effect on the other inhibitor.

Other groups have also investigated the strategy of linking HIV inhibitors in order to increase effectiveness. One very effective linked compound is a CD4 antibody having each heavy chain extended with a gp-41-binding C-peptide. This chimeric protein showed HIV inhibition with an IC₅₀ as low as 14 pM. This group also produced another excellent compound by linking a CCR5 antibody with a C-peptide. In both cases, the antibody was expressed having one C-peptide at the C-terminus of each heavy chain of the antibody, resulting in a ratio of 2:1 C peptide: antibody. While both of these chimeric compounds would likely be highly effective microbicides, they have the disadvantage of being produced in a mammalian cell system (HEK cells), which would make it difficult to produce gram-quantities of the inhibitor. In this system, Griff37 was produced from E. coli in shaker flasks and could likely be produced in large quantities by fermentation or in a plant based expression system, both of which were recently shown to be useful in making large quantities of functional griffithsin. Another effective HIV inhibitor was produced by linking two domains from CD4 with a single chain variable region from the antibody 17b, which is known to interact with gp120 on a site near its CCR5-binding region. This inhibitor was shown to be effective at nanomolar levels and was expressed by recombinant vaccinia virus. A recent study of a gp120 antibody in various monomer/dimeric covalent configurations and a gp41 antibody in these configurations also showed the importance of multivalency in HIV inhibition.

Structurally, many questions remain unanswered about the compounds in the present study. Griffithsin consistently crystallizes as a dimer, which could imply that a linked compound may include two molecules of griffithsin and two molecules of C-peptide. However, the concentrations used in the functional assays are orders of magnitude lower than those for structural studies, so it has not yet been determined if the griffithsin dimer is a consideration in the function of the compounds reported here.

The ¹H, ¹⁵N correlation spectrum of griffithsin alone shows a nicely dispersed set of peaks indicative of a fully folded protein (FIG. 9a). To Applicants' knowledge, this is the first NMR spectrum reported of griffithsin, although several high quality X-ray structures have been determined. The benefit of NMR spectra is that comparisons can quickly and easily be made between variants of a protein without the need for a full structural determination if sequence assignments have been completed. Such work for griffithsin is ongoing.

NMR spectra of the strategically linked compounds show some resonances to indicate that both Griff37 and C37-linker-CD4M33_{C1F23} are folded, although it is not clear what percentage of each sample is folded. The strong resonances in the unfolded region could simply be due to the presence of disordered C37 peptide and linker, or could in addition have a significant component from unfolded (and presumably non functional) protein. In the latter case, small improvements to the refolding procedure could lead to significantly more potent inhibitors.

The strategically linked compounds reported here show picomolar levels of activity in many assays, but may still be able to be improved. For example, the C-peptide component may be able to be improved by capping the charged ends that occur naturally when a protein is expressed rather than synthesized. In here, N-acetylated, C-amidated C37 inhibits an R5 fusion assay with an IC₅₀ of 18.2 nM, as reported above. However, C37 without the terminal modifications inhibits with an IC₅₀ of >100 nM. Therefore, it is possible that capping the C-terminal end of Griff37 and C37CD4M33_{C1F23} could increase the potency of these compounds. In addition, others have reported further improvements in C-peptides that increase their potency, which may be able to be incorporated into the strategy presented here.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

### REFERENCES

1. UNAIDS, 2008 Report on the Global AIDS epidemic. 2008, WHO/UNAIDS.
2. Sullivan, N., et al., CD4-Induced conformational changes in the human immunodeficiency virus type 1 gp120 glycoprotein: consequences for virus entry and neutralization. J Virol, 1998. 72(6): p. 4694-703.
3. Sattentau, Q. J., et al., Conformational changes induced in the envelope glycoproteins of the human and simian immunodeficiency viruses by soluble receptor binding. J Virol, 1993. 67(12): p. 7383-93.
4. Kwong, P. D., et al., Structure of an HIV gp120 envelope glycoprotein in complex with the CD4 receptor and a neutralizing human antibody. [see comments]. Nature, 1998. 393(6686): p. 648-59.
5. Berger, E. A., P. M. Murphy, and J. M. Farber, Chemokine receptors as HIV-1 coreceptors: roles in viral entry, tropism, and disease. Annu Rev Immunol, 1999. 17: p. 657-700.
6. Root, M. J. and H. K. Steger, HIV-1 gp41 as a target for viral entry inhibition. 2004: p. 1805-25.
7. Eckert, D. M. and P. S. Kim, Mechanisms of viral membrane fusion and its inhibition. Annu Rev Biochem, 2001. 70: p. 777-810.
8. Miyauchi, K., et al., HIV enters cells via endocytosis and dynamin-dependent fusion with endosomes. Cell, 2009. 137(3): p. 433-44.
9. Hart, C. E. and T. Evans-Strickfaden, HIV-1 entry inhibitors as microbicides, in Entry Inhibitors in HIV Therapy, J. D. Reeves and C. A. Derdeyn, Editors. 2007, Birkhauser Verlag. p. 99-117.
10. Jacobson, J. M., et al., Treatment of advanced human immunodeficiency virus type 1 disease with the viral entry inhibitor PRO 542. Antimicrob Agents Chemother, 2004. 48(2): p. 423-9.
11. Wang, H. G., R. E. Williams, and P. F. Lin, A novel class of HIV-1 inhibitors that targets the viral envelope and inhibits CD4 receptor binding. Curr Pharm Des, 2004. 10(15): p. 1785-93.
12. Mori, T., et al., Isolation and characterization of griffithsin, a novel HIV-inactivating protein, from the red alga Griffithsia sp. J Biol Chem, 2005. 280(10): p. 9345-53.
13. Emau, P., et al., Griffithsin, a potent HIV entry inhibitor, is an excellent candidate for anti-HIV microbicide. J Med Primatol, 2007. 36(4-5): p. 244-53.
14. O'Keefe, B. R., et al., Scaleable manufacture of HIV-1 entry inhibitor griffithsin and validation of its safety and efficacy as a topical microbicide component. Proc Natl Acad Sci USA, 2009. 106(15): p. 6099-104.
15. Ziolkowska, N. E., et al., Domain-swapped structure of the potent antiviral protein griffithsin and its mode of carbohydrate binding. Structure, 2006. 14(7): p. 1127-35.
16. Ziolkowska, N. E., et al., Crystallographic studies of the complexes of antiviral protein griffithsin with glucose and N-acetylglucosamine. Protein Sci, 2007. 16(7): p. 1485-9.
17. Martin, L., et al., Rational design of a CD4 mimic that inhibits HIV-1 entry and exposes cryptic neutralization epitopes. Nat Biotechnol, 2003. 21(1): p. 71-6.
18. Champagne, K., A. Shishido, and M. J. Root, Interactions of HIV-1 inhibitory peptide T20 with the gp41 N-HR coiled coil. J Biol Chem, 2009. 284(6): p. 3619-27.
19. Liu, S., et al., Different from the HIV fusion inhibitor C34, the anti-HIV drug Fuzeon (T-20) inhibits HIV-1 entry by targeting multiple sites in gp41 and gp120. J Biol Chem, 2005. 280(12): p. 11259-73.
20. Poveda, E., V. Briz, and V. Soriano, Enfuvirtide, the first fusion inhibitor to treat HIV infection. AIDS Reviews, 2005. 7(3): p. 139-47.
21. Lu, M. and P. S. Kim, A trimeric structural subdomain of the HIV-1 transmembrane glycoprotein. J Biomol Struct Dyn, 1997. 15(3): p. 465-71.
22. Root, M. J., M. S. Kay, and P. S. Kim, Protein design of an HIV-1 entry inhibitor. Science, 2001. 291(5505): p. 884-8.
23. Chan, D. C., C. T. Chutkowski, and P. S. Kim, Evidence that a prominent cavity in the coiled coil of HIV type 1 gp41 is an attractive drug target. Proceedings of the National Academy of Sciences of the United States of America, 1998. 95(26): p. 15613-7.
24. Pleskoff, O., et al., Identification of a chemokine receptor encoded by human cytomegalovirus as a cofactor for HIV-1 entry. Science, 1997. 276(5320): p. 1874-8.
25. Shu, W., et al., Helical interactions in the HIV-1 gp41 core reveal structural basis for the inhibitory activity of gp41 peptides. Biochemistry, 2000. 39(7): p. 1634-42.
26. Martin, L., et al., Structural and functional analysis of the RANTES-glycosaminoglycans interactions. Biochemistry, 2001. 40(21): p. 6303-18.
27. Melikyan, G. B., et al., Evidence that the transition of HIV-1 gp41 into a six-helix bundle, not the bundle configuration, induces membrane fusion. J Cell Biol, 2000. 151(2): p. 413-23.
28. Jin, H., et al., Structural and functional studies of the potent anti-HIV chemokine variant P2-RANTES. Proteins, 2009.
29. Clore, G. M. and A. M. Gronenborn, NMR structures of proteins and protein complexes beyond 20,000 M(r). Nat Struct Biol, 1997. 4 Suppl: p. 849-53.
30. LiWang, A. C., et al., Dynamics Study on the anti-human immunodieficiency virus chemokine viral macrophage inflammatory protein-II (vMIP-II) reveals a fully monomeric protein. Biochemistry, 1999. 38: p. 442-453.
31. Frank, M. K., Clore., G. M., and Gronenborn, A. M., Structural and dynamic characterization of the urea denatured state of the immunoglobulin binding domain of streptpcoccal protein G by multidimensional heteronuclear NMR spectroscopy. Protein Sci., 1995. 4: p. 2605-2615.
32. Lu, M., S. C. Blacklow, and P. S. Kim, A trimeric structural domain of the HIV-1 transmembrane glycoprotein. Nature Structural Biology, 1995. 2(12): p. 1075-82.
33. Stoddart, C. A., et al., Albumin-conjugated C34 peptide HIV-1 fusion inhibitor: equipotent to C34 and T-20 in vitro with sustained activity in SCID-hu Thy/Liv mice. J Biol Chem, 2008. 283(49): p. 34045-52.
34. Jekle, A., et al., CD4-BFFI: a novel, bifunctional HIV-1 entry inhibitor with high and broad antiviral potency. Antiviral Res, 2009. 83(3): p. 257-66.
35. Ji, C., et al., CD4-anchoring HIV-1 fusion inhibitor with enhanced potency and in vivo stability. J Biol Chem, 2009. 284(8): p. 5175-85.
36. Kopetzki, E., et al., Closing two doors of viral entry: intramolecular combination of a coreceptor- and fusion inhibitor of HIV-1. Virol J, 2008. 5: p. 56.
37. Giomarelli, B., et al., Recombinant production of anti-HIV protein, griffithsin, by auto-induction in a fermentor culture. Protein Expr Purif, 2006. 47(1): p. 194-202.
38. Dey, B., C. S. Del Castillo, and E. A. Berger, Neutralization of human immunodeficiency virus type 1 by sCD4-17b, a single-chain chimeric protein, based on sequential interaction of gp120 with CD4 and coreceptor. J Virol, 2003. 77(5): p. 2859-65.
39. Klein, J. S., et al., Examination of the contributions of size and avidity to the neutralization mechanisms of the anti-HIV antibodies b12 and 4E10. Proc Natl Acad Sci USA, 2009. 106(18): p. 7385-90.
40. Deng, Y., et al., Protein design of a bacterially expressed HIV-1 gp41 fusion inhibitor. Biochemistry, 2007. 46(14): p. 4360-9.
41. Ziolkowska, N. E., et al., Crystallographic, thermodynamic, and molecular modeling studies of the mode of binding of oligosaccharides to the potent antiviral protein griffithsin. Proteins, 2007. 67(3): p. 661-70.
42. Wishart, D. S., et al., ¹H, ¹³C, ¹⁵N chemical shift referencing in biomolecular NMR. J. Biomol. NMR, 1995. 6: p. 135-140.
43. Delaglio, F., et al., NMRPipe: A multidimensional spectral processing system based on UNIX pipes. J. Biomol. NMR, 1995. 6: p. 277-293.

1. An isolated chimeric polypeptide comprising a first portion comprising SEQ ID NO. 2, amino acid residues 11 to 101 of SEQ ID NO. 2, a substantial homologue of either one thereof, or an amino acid sequence that is at least about 80% identical to SEQ ID NO. 2 and a second portion selected from a gp41-binding protein, a CCR5-binding protein, a gp120-binding protein, SEQ ID NO. 2, amino acid residues 11 to 101 of SEQ ID NO. 2 or a substantial homologue of any one thereof.

2. The isolated chimeric polypeptide of claim 1, wherein the second portion is a gp120-binding protein and the gp41-binding protein is selected from C37, C34, C52L, T-2635, T20 or a substantial homologue thereof.

3. The isolated chimeric polypeptide of claim 1, wherein the second portion is a CCR5-binding protein and the CCR5-binding protein is selected from RANTES, P2-RANTES, PSC-RANTES, 5P12-RANTES, 5P14-RANTES, 6P4-RANTES, MIP-1α, MIP-1β, U83A, a CCR5 antibody or a substantial homologue of any one thereof.

4. The isolated chimeric polypeptide of claim 1, wherein the second portion is a gp120-binding protein and the gp120-binding protein is selected from cyanovirin-N (CVN), 12p1, CD4M33, CD4M47 or a substantial homologue of any one thereof.

5. The isolated chimeric polypeptide of claim 1, further comprising a peptide linker between the first portion and the second portion.

6. An isolated polypeptide comprising an amino acid sequence of SEQ ID NO. 3, 4 or 5, or an amino acid sequence having at least 80% identity to SEQ ID NO. 3, 4 or 5.

7. A peptide conjugate comprising a carrier covalently or non-covalently linked to an isolated chimeric polypeptide of claim 1.

8. A composition comprising a carrier and an isolated chimeric polypeptide of claim 1.

9. An antibody or antibody fragment that binds an isolated chimeric polypeptide of claim 1.

10. A polynucleotide encoding the chimeric polypeptide of claim 1 or the antibody or the antibody fragment of claim 9.

11. A composition comprising a first polypeptide comprising SEQ ID NO. 2, amino acid residues 11 to 101 of SEQ ID NO. 2, a substantial homologue of either one thereof or an amino acid sequence that is at least about 80% identical to SEQ ID NO. 2 and a second polypeptide selected from a gp41-binding protein, a CCR5-binding protein, a gp120-binding protein, a chimeric polypeptide comprising two different proteins selected from a gp41-binding protein, a CCR5-binding protein or a gp120-binding protein, or a substantial homologue of any one thereof.

12. The composition of claim 11, wherein the first polypeptide and the second polypeptide are present in a mole ratio of about 1:10 to about 10:1, or 1:5 to 5:1 or 1:1.

13. A method for preventing or inhibiting HIV entry into a cell, comprising contacting the cell with an effective amount of an isolated chimeric polypeptide of claim 1.

14. A method for inhibiting HIV infection in a subject, comprising administering to the subject an effective amount of an isolated chimeric polypeptide of claim 1.

15. The method of claim 14, wherein the subject is infected with HIV or is at risk of HIV infection.

16. An isolated chimeric polypeptide comprising a first portion comprising a gp41-binding protein and a second portion comprising a gp120-binding protein.

17. The isolated chimeric polypeptide of claim 15, wherein the gp41-binding protein is one or more of an anti-gp41 antibody, antibody fragment or derivative thereof, a C-peptide, a N-peptide, C37; C-37ac; C37(Q652L); N-acetylated, C-term amidated C37; N-acetylated, C-term amidated C37(Q652L); C34, C52L; T-2635; T20, N-peptides, N17, N23, N36 or a substantial homologue thereof.

18. The isolated chimeric polypeptide of claim 15, wherein the gp120-binding protein is an anti-gp120 binding antibody, antibody fragment or derivative thereof, cyanovirin-N (CVN), 12p1, CD4M33, CD4M47, CD4M33_{C1F23}, C37CD4M33_{C1F23}, actinohivin, or a substantial homologue of any one thereof.

19. The isolated chimeric polypeptide of claim 15, further comprising a peptide linker between the first portion and the second portion.

20. The isolated chimeric polypeptide of claim 15, further comprising an anti-HIV small molecule or compound bound to the isolated chimeric polypeptide.

21. A method for preventing or inhibiting HIV entry into a cell, comprising contacting the cell with an effective amount of a chimeric polypeptide of claim 15.
